(19) United States
(12) Patent Application Publication
(10) Pub. No.: US 2012/0100539 A1
(43) Pub. Date: Apr. 26, 2012

(54) METHOD FOR DETERMINING RISK FOR KIDNEY STONES DEVELOPING OR RECURRING AND METHOD FOR USING SINGLE-NUCLEOTIDE POLYMORPHISM RS12313273 AS BIOMARKER FOR DETERMINING DEVELOPMENT OR RECURRENCE OF KIDNEY STONE

(75) Inventors: Wei-Chiao Chang, Kaohsiung City (TW); Yii-Her Chou, Kaohsiung City (TW); Suh-Hang Hank Juo, Kaohsiung City (TW); Mu-En Liu, Kaohsiung City (TW); Chen-Chia Chang, Kaohsiung City (TW); Yi-Ching Chiu, Kaohsiung (TW)

(73) Assignee: KAOHSIUNG MEDICAL UNIVERSITY, Kaohsiung City (TW)

(21) Appl. No.: 13/277,225

(22) Filed: Oct. 20, 2011

(30) Foreign Application Priority Data
Oct. 20, 2010 (TW) 099135698

(51) Int. Cl. C12Q 1/68

(52) U.S. Cl. 435/6.11

(57) ABSTRACT

The invention provides a method for determining a risk for kidney stones to develop in a subject, including: obtaining a biosample of the subject; detecting the presence of the single-nucleotide polymorphism rs12313273 (C/T) at position 30881 of the ORAI1 gene (SEQ ID No.: 1) in the biosample; and determining a risk for kidney stones to develop in the subject, wherein if the presence of a C allele of the single-nucleotide polymorphism rs12313273 (C/T) is detected, it indicates that the subject has an increased risk for kidney stones to develop.

### CROSS REFERENCE TO RELATED APPLICATIONS

This Application claims priority of Taiwan Patent Application No. 099135698, filed on Oct. 20, 2010, the entirety of which is incorporated by reference herein.

### INCORPORATION BY REFERENCE OF SEQUENCE LISTING

A sequence listing submitted as a text file via EFS-Web is incorporated herein by reference. The text file containing the sequence listing is named “0911-A52112-US_Seq_Listing.txt”; its date of creation is Mar. 15, 2011; and its size is 8,2232 bytes.

### BACKGROUND OF THE INVENTION

1. Field of the Invention

The present invention relates to a method for determining a risk of a kidney stone developing and/or recurring, and in particular relates to a method for using a single-nucleotide polymorphism rs12313273 as a biomarker for development and/or recurrence of kidney stones.

2. Description of the Related Art

A calcium containing stone, a common kind of kidney stones disease, belongs to diseases co-influenced by multiple factors. Urinary tract obstruction caused by kidney stones usually result in colic, infection, hydronephrosis, etc., which seriously influences a quality of life of a patient. For all kidney stone patients, about 25% have a family medical history of kidney stones.

A family member with a family medical history of kidney stones may more easily suffer from kidney stones, wherein the morbific age thereof may be younger and the probability for recurrence after treatment may be higher. According to the above-mentioned, it is known that a gene composition is not only an important pathogenic factor for kidney stones, but is also significantly related to the severity and recurrence rate of kidney stones disease.

The ORAI1 gene is a subunit of a channel protein of a store-operated calcium channel. For non-activated cells, such as immune T cells, epithelium cells and mast cells, the store-operated Ca²⁺ channel is a meanest approach for Ca²⁺ entering therein. Change of calcium ion concentrations in a cell affects changes for many reactions in a cell, such as inflammation, cell proliferation and apoptosis, etc. The article (Feske, S., Gwack, Y., Prakriya, M. et al.: A mutation in Orail causes immune deficiency by abrogating CRAC channel function. Nature 2006; 441: 179) discloses that ORAI1 gene mutation in T cells results in non-equilibrium for calcium ions and thus further results in serious immune diseases. In mast cells, ORAI1-mediated ion channel is capable of changing an amount of calcium ion entering into the cells and further participates in an allergic response.

A calcium containing stone endangers the subsistence of kidney cells and influence calcium ion equilibrium therein to result in inflammation and apoptosis. Calcium containing stones deteriorate and cause the formation of other calcium containing stones. In order for early prevention of the development of calcium containing stones, an effect for the single-nucleotide polymorphism of the gene for a store-operated calcium channel on development of calcium containing stones is investigated in the invention. The invention is able to improve the understanding for the morbific mechanisms of calcium containing stones, and the invention may be used to detect whether a subject has calcium containing stones and may be used to develop a diagnostic reagent.

### BRIEF SUMMARY OF THE INVENTION

The invention provides a method for determining a risk for kidney stones to develop in a subject, comprising: obtaining a biosample of the subject; detecting the presence of the single-nucleotide polymorphism rs12313273 (C/T) at position 30881 of the ORAI1 gene (SEQ ID No.: 1) in the biosample; and determining a risk for kidney stones to develop in the subject, wherein if the presence of a C allele of the single-nucleotide polymorphism rs12313273 (C/T) is detected, it indicates that the subject has an increased risk for kidney stones to develop.

The invention also provides a method for determining a risk for kidney stones to reoccur in a subject suffering from or once suffering from kidney stones, comprising: obtaining a biosample of the subject suffering from or once suffering from kidney stones; detecting the presence of the single-nucleotide polymorphism rs12313273 (C/T) at position 30881 of the ORAI1 gene (SEQ ID No.: 1) in the biosample; and determining a risk for kidney stones to reoccur in the subject suffering from or once suffering from kidney stones, wherein if the presence of a C allele of the single-nucleotide polymorphism rs12313273 (C/T) is detected, it indicates that the subject suffering from or once suffering from kidney stones has an increased risk for kidney stones to reoccur.

The invention further provides a method for using a single-nucleotide polymorphism rs12313273 (SEQ ID No.: 3) as a biomarker for determining the development of kidney stones.

The invention further provides a method for using a single-nucleotide polymorphism rs12313273 (SEQ ID No.: 3) as a biomarker for determining the recurrence of kidney stones.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows the relative positions of five single-nucleotide polymorphisms, rs12320939, rs12313273, rs7135617, rs6486795 and rs712853, in the ORAI1 gene.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

In order to realize the relationship between the single-nucleotide polymorphism (SNP) and kidney stones, an effect of the single-nucleotide polymorphism of the gene for a store-operated Ca²⁺ channel on development of kidney stones is investigated in the invention. In one embodiment, the ORAI1 gene (SEQ ID No.: 1, wherein the position 32519 thereof is the transcription +1 position.) is selected for investigation.

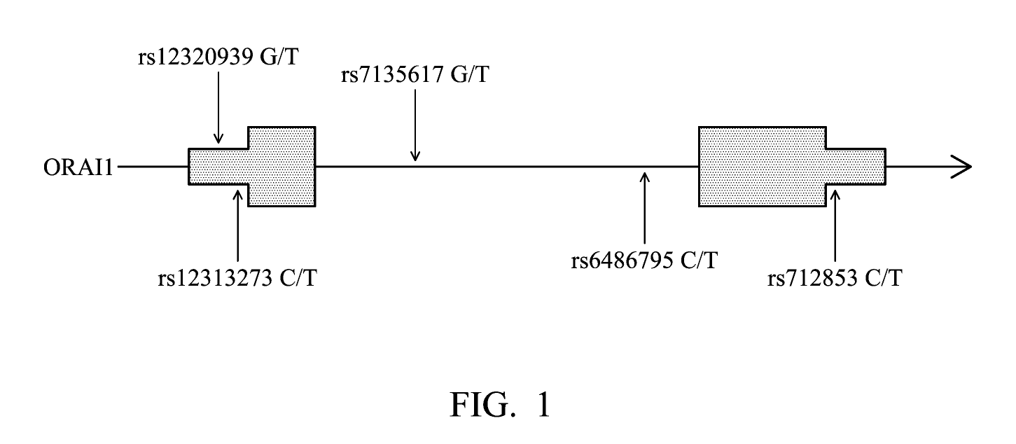

In one embodiment, five single-nucleotide polymorphisms of the ORAI1 gene are selected and the individual relationships between the selected single-nucleotide polymorphisms and development of kidney stones are analyzed. The five single-nucleotide polymorphisms of the ORAI1 gene are rs12320939 (SEQ ID No.: 2 is at position 30567 to position 30618 of the SEQ ID No.: 1; according to the transcription +1 position of SEQ ID No.: 1 as a standard, SEQ ID No.: 2 is identified as from −1952 position to −1901 position in the SEQ ID No.: 1.), rs12313273 (SEQ ID No.: 3 is at position 30855 to position 30906 of the SEQ ID No.: 1; according to the transcription +1 position of SEQ ID No.: 1 as a standard, SEQ ID No.: 3 is identified as from −1664 position to −1613 position in the SEQ ID No.: 1.), rs7135617 (SEQ ID No.: 4 is at position 36850 to position 36901 of the SEQ ID No.: 1; according to the transcription +1 position of SEQ ID No.:1 as a standard, SEQ ID No.: 4 is identified as from 4332 position to 4383 position in the SEQ ID No.: 1.), rs6486795 (SEQ ID No.: 5 is at position 43762 to position 43813 of the SEQ ID No.: 1; according to the transcription +1 position of SEQ ID No.: 1 as a standard, SEQ ID No.: 5 is identified as from 11244 position to 11295 position in the SEQ ID No.: 1.) and rs712853 (SEQ ID No.: 6 is at position 47514 to position 47565 of the SEQ ID No.: 1; according to the transcription +1 position of SEQ ID No.: 1 as a standard, SEQ ID No.: 6 is identified as from 14996 position to 15047 position in the SEQ ID No.: 1.). The relative positions of the five single-nucleotide polymorphisms mentioned above in the ORAI1 gene are shown in FIG. 1.

rs12320939 (G/T) is at position 30593 of the SEQ ID No.: 1, rs12313273 (C/T) is at position 30881 of the SEQ ID No.: 1, rs7135617 (G/T) is at position 36876 of the SEQ ID No.: 1, rs6486795 (C/T) is at position 43788 of the SEQ ID No.: 1 and rs712853 (C/T) is at position 47593 of the SEQ ID No.: 1.

In one embodiment, determinations for genotypes of the five single-nucleotide polymorphisms mentioned above are performed to biosamples of a healthy group and kidney stone group, respectively, and the results of the determinations are analyzed by statistics to confirm that rs12313273 (C/T) is a potent biomarker for determining the development of kidney stones. In another embodiment, determinations for genotypes of the five single-nucleotide polymorphisms mentioned above are performed to biosamples of a group of patients suffering from or once suffering from kidney stones, wherein the number of patients having recurring kidney stones was recorded and the genotypes of all patients were also recorded, respectively, and the results of the determinations are analyzed by statistics to confirm that rs12313273 (C/T) is a potent biomarker for determining the recurrence of kidney stones.

Therefore, in one aspect of the invention, the invention relates to a method for determining a risk for kidney stones to develop in a subject comprising the steps as described in the following. First, a biosample of a subject is obtained. The subject may comprise a mammal. In one embodiment, the subject may comprise a human. The biosample of the invention may be collected or isolated from any source containing chromosomal DNA. The source may comprise blood or saliva. In one embodiment, the biosample may comprise blood or saliva, for example, about 3-5 c.c. of blood or saliva.

Then, the presence of the single-nucleotide polymorphism rs12313273 (C/T) at position 30881 of the ORAI1 gene (SEQ ID No.:1) in the biosample is detected. A method for detecting the single-nucleotide polymorphism rs12313273 (C/T) may comprise primer extension (such as PinPoint assay, Massextend™, SPC-SBE, or GOOD assay), hybridization (such as TaqMan assay, bead array, or single-nucleotide polymorphism chip), ligation (combinatorial fluorescence energy transfer (CFET) tags), and enzymatric cleavage (RFLP, Invader® assay), PCR-SSCP (single-strand conformation polymorphism), MRD (mismatch repair dection), BeadArray™, or SNPlex™. In one embodiment, a genotype of the single-nucleotide polymorphism rs12313273 may be identified with a TaqMan assay. In one embodiment, the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

Finally, a risk for kidney stones to develop in the subject is determined. In one embodiment, the kidney stones may comprise calcium kidney stones. According to the result of the detection mentioned above, if the presence of a C allele of the single-nucleotide polymorphism rs12313273 (C/T) is detected, it indicates that the subject has an increased risk for kidney stones to develop.

In one embodiment, the risk for kidney stones to develop in the subject is increased with an odds ratio of at least about 1.4-1.6, preferably 1.56 when the C allele of the single-nucleotide polymorphism rs12313273 (C/T) is detected in the biosample as opposed to the T allele of the single-nucleotide polymorphism rs12313273 (C/T) being detected in the biosample. In another embodiment, the risk for kidney stones to develop in the subject is increased with an odds ratio of at least about 1.7-1.9, preferably 1.82 when the genotype CT (one allele is C allele and the other is T allele) of the single-nucleotide polymorphism rs12313273 (C/T) is detected in the biosample as opposed to the genotype TT (both alleles are T alleles) of the single-nucleotide polymorphism rs12313273 (C/T) being detected in the biosample. In further another embodiment, the risk for kidney stones to develop in the subject is increased with an odds ratio of at least about 2.0-2.2, preferably 2.10 when the genotype CC (both alleles are C alleles) of the single-nucleotide polymorphism rs12313273 (C/T) is detected in the biosample as opposed to the genotype TT of the single-nucleotide polymorphism rs12313273 (C/T) being detected in the biosample.

In other words, as compared with the subject with the genotype TT of the single-nucleotide polymorphism rs12313273 (C/T), the subject with the genotype CT of the single-nucleotide polymorphism rs12313273 (C/T) has the higher risk for kidney stones to develop. Furthermore, as compared with the subject with the genotype CT of the single-nucleotide polymorphism rs12313273 (C/T), the subject with the genotype CC of the single-nucleotide polymorphism rs12313273 (C/T) has the higher risk for kidney stones to develop. Namely, as compared with the subject with the genotype TT or CT of the single-nucleotide polymorphism rs12313273 (C/T), the subject with the genotype CC of the single-nucleotide polymorphism rs12313273 (C/T) has the highest risk for kidney stones to develop.

According to the above-mentioned, it is clearly understood that the single-nucleotide polymorphism rs12313273 (C/T) may be used as one of the biomarkers of development of kidney stones.

Therefore, in another aspect of the invention, the invention relates to a method for using a single-nucleotide polymorphism rs12313273 (SEQ ID No.: 3) as a biomarker for determining the development of kidney stones or a method for using a single-nucleotide polymorphism rs12313273 (SEQ ID No.: 3) as a biomarker for determining a risk for kidney stones to develop in a subject.

In addition, in further another aspect of the invention, the invention relates to a method for determining a risk for kidney stones to reoccur in a subject suffering from or once suffering from kidney stones comprising the steps as described in the following. First, a biosample of a subject suffering from or once suffering from kidney stones is obtained. The subject suffering from or once suffering from kidney stones may comprise a mammal. In one embodiment, the subject mentioned above may comprise a human. The biosample mentioned above may be collected or isolated from any source containing chromosomal DNA. The source may comprise blood or saliva. In one embodiment, the biosample may comprise blood or saliva, for example, about 3-5 c.c. of blood or saliva.

Then, the presence of the single-nucleotide polymorphism rs12313273 (C/T) at position 30881 of the ORAI1 gene (SEQ ID No.: 1) in the biosample is detected. A method for detecting the single-nucleotide polymorphism rs12313273 (C/T) may comprise a primer extension (such as PinPoint assay, Massextend™, SPC-SBE, or GOOD assay), hybridization (such as TaqMan assay, bead array, or single-nucleotide polymorphism chip), ligation (combinatorial fluorescence energy transfer (CFET) tags), and enzymatric cleavage (RFLP, Invader® assay), PCR-SSCP (single-strand conformation polymorphism), MRD (mismatch repair dection), BeadArray™, or SNPlex™. In one embodiment, a genotype of the single-nucleotide polymorphism rs12313273 may be identified with a TaqMan assay. In one embodiment, the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

Finally, a risk for kidney stones to reoccur in a subject suffering from or once suffering from kidney stones is determined. In one embodiment, the kidney stones may comprise calcium kidney stones. According to the result of the detection mentioned above, if the presence of a C allele of the single-nucleotide polymorphism rs12313273 (C/T) is detected, it indicates that the subject suffering from or once suffering from kidney stones has an increased risk for kidney stones to reoccur.

In one embodiment, the subject suffering from or once suffering from kidney stones has an increased risk for kidney stones to recur when the genotype CC or CT of the single-nucleotide polymorphism rs12313273 (C/T) is detected in the biosample as opposed to the genotype TT of the single-nucleotide polymorphism rs12313273 (C/T) being detected in the biosample. In another embodiment, the subject suffering from or once suffering from kidney stones has an increased risk for kidney stones to recur when the genotype CC of the single-nucleotide polymorphism rs12313273 (C/T) is detected in the biosample as opposed to the genotype CT of the single-nucleotide polymorphism rs12313273 (C/T) being detected in the biosample. In further another embodiment, the subject suffering from or once suffering from kidney stones has an increased risk for kidney stones to recur when the genotype CC of the single-nucleotide polymorphism rs12313273 (C/T) is detected in the biosample as opposed to the genotype TT of the single-nucleotide polymorphism rs12313273 (C/T) being detected in the biosample.

In other words, as compared with the subject suffering from or once suffering from kidney stones with the genotype TT of the single-nucleotide polymorphism rs12313273 (C/T), the subject suffering from or once suffering from kidney stones with the genotype CT of the single-nucleotide polymorphism rs12313273 (C/T) has the higher risk for kidney stones to reoccur. Furthermore, as compared with the subject suffering from or once suffering from kidney stones with the genotype CT of the single-nucleotide polymorphism rs12313273 (C/T), the subject suffering from or once suffering from kidney stones with the genotype CC of the single-nucleotide polymorphism rs12313273 (C/T) has the higher risk for kidney stones to reoccur. Namely, as compared with the subject suffering from or once suffering from kidney stones with the genotype TT or CT of the single-nucleotide polymorphism rs12313273 (C/T), the subject suffering from or once suffering from kidney stones with the genotype CC of the single-nucleotide polymorphism rs12313273 (C/T) has the highest risk for kidney stones to reoccur.

In one example, the risk for kidney stones to reoccur in the subject suffering from or once suffering from kidney stones is increased with an odds ratio of at least about 2.2-2.4, preferably 2.31 when the genotype CT (one allele is C allele and the other is T allele) of the single-nucleotide polymorphism rs12313273 (C/T) is detected in the biosample as opposed to the genotype TT (both alleles are T alleles) of the single-nucleotide polymorphism rs12313273 (C/T) being detected in the biosample. Moreover, the risk for kidney stones to reoccur in the subject suffering from or once suffering from kidney stones is increased with an odds ratio of at least about 3.6-3.8, preferably 3.73 when the genotype CC (both alleles are C alleles) of the single-nucleotide polymorphism rs12313273 (C/T) is detected in the biosample as opposed to the genotype TT of the single-nucleotide polymorphism rs12313273 (C/T) being detected in the biosample.

According to the above-mentioned, it is clearly understood that the single-nucleotide polymorphism rs12313273 (C/T) may be used as one of the biomarkers of recurrence of kidney stones.

Therefore, in further another aspect of the invention, the invention relates to a method for using a single-nucleotide polymorphism rs12313273 (SEQ ID No.: 3) as a biomarker for determining the recurrence of kidney stones or a method for using a single-nucleotide polymorphism rs12313273 (SEQ ID No.: 3) as a biomarker for determining a risk for kidney stones to reoccur in a subject suffering from or once suffering from kidney stones.

### EXAMPLES

Relationship Between the Single-Nucleotide Polymorphism (SNP) and Kidney Stone

1. Material and Method

(a) DNA Extraction for Biosample of a Subject

According the standard procedure provided by the Puregene™ kit from Gentra (Research Triangle, NC), 5 ml of venous whole blood was placed in a tube containing EDTA and centrifuged at 3000 rpm/min for 10 minutes to separate plasma, leukocyte buffy coat and erythrocytes. The buffy coat was taken out from the tube and added into a 1.5 ml microcentrifuge tube by a micropipette and three times that of the buffy coat volume of a RBC lysis solution was added into the microcentrifuge tube. The microcentrifuge tube was shaken to mix the buffer and the buffy coat well, and centrifuged at 13,000-16,000 rpm/min for 1 minute to precipitate the leukocyte containing DNA. Then, a supernatant therefrom was removed from the microcentrifuge tube and 600 μl of a cell lysis buffer was added thereto, and then the microcentrifuge tube was shaken to mix the remainder and the cell lysis buffer well and kept for least one night. After the cell membrane was broken and DNA was released from the cells, 300 μl of a protein precipitation solution was added into the microcentrifuge tube and mixed with the remainder. Next, the microcentrifuge tube was centrifuged at 13,000-16,000 rpm/min for 5 minutes to precipitate impurities therein. A supernatant containing DNA therefrom was taken and added into a new microcentrifuge tube and an equal amount of isopropanol was added thereto and the new microcentrifuge tube was shaken, slightly. After that, the new microcentrifuge tube was centrifuged at 13,000-16,000 rpm/min for 2 minutes to obtain a white precipitate containing DNA and a supernatant. The supernatant was removed carefully and 450 μl of ethanol was added into the microcentrifuge tube. Then, the new microcentrifuge tube was centrifuged at 13,000-16,000 rpm/min for 3 minutes and a supernatant therefrom was removed to remove the undesired salts. The ethanol remaining in the new microcentrifuge tube was dried to completely evaporate and then 100 μl of d. d. water was added into the new microcentrifuge tube. Then, the new microcentrifuge tube was placed in a 65° C. water bath for 5 minutes to accelerate redissolving of the DNA in the d. d. water.

The concentration and purity (O.D. 260/280) of the obtained DNA was determined by a spectrophotometer and then diluted to a concentration of 10 ng/μl for use in the determination of a genotype.

(b) Selection Conditions for Single-Nucleotide Polymorphisms

The single-nucleotide polymorphisms of the invention were selected from the data base Phase III of the International Haplotype HapMap project (http://www.hapmap.org). The Haplotype HapMap project was co-established by the governments of the United State of America, China, Canada, Japan, Nigeria and Britain, for the purpose of identifying and cataloging genetic information of human beings, such as those related to single-nucleotide polymorphisms, from different ethnic groups, populations, and geographic locations. The Haplotype HapMap project encompasses Nigerian-Americans, Japanese-Americans, Chinese-Americans and European-Americans, wherein the frequencies for single-nucleotide polymorphisms distributed among different populations can be accessed.

The selection standard for the single-nucleotide polymorphisms in the invention is that the single-nucleotide polymorphism must meet r²≧0.8 and the minor allele frequency (MAF) for Han Chinese in Beijing≧10%, wherein the selected single-nucleotide polymorphisms are all representative tagging single-nucleotide polymorphisms (tSNPs) in linkage disequilibrium in the genomic regions. According to the standard, five tagging single-nucleotide polymorphisms were selected from the ORAI1 gene (SEQ ID. No.: 1) in the invention. The five tagging single-nucleotide polymorphisms were rs12320939 (SEQ ID No.: 2 is at position 30567 to position 30618 of the SEQ ID No.: 1), rs12313273 (SEQ ID No.: 3 is at position 30855 to position 30906 of the SEQ ID No.: 1), rs7135617 (SEQ ID No.: 4 is at position 36850 to position 36901 of the SEQ ID No.: 1), rs6486795 (SEQ ID No.: 5 is at position 43762 to position 43813 of the SEQ ID No.: 1) and rs712853 (SEQ ID No.: 6 is at position 47514 to position 47565 of the SEQ ID No.: 1), respectively.

(c) Genotype Determination

The TaqMan technique (TaqMan Genotyping Assays products, Applied Biosystems Inc.) was used to perform the genotype determination in the invention. In the TaqMan technique, two probes (Minor Grove Binder probe) carrying different fluorescent dyes (FAM™ dye and VIC™ dye) at their 5′ ends, respectively are used. Due to the difference of the designed sequences for the two probes, the two probes are able to match with the two different alleles of a single-nucleotide polymorphism, respectively. In addition, a non-fluorescent quencher (NFQ) at the 3′ end of the respective probe is used to absorb the fluorescence energy of the fluorescent dye so that no fluorescent light was released from the fluorescent dye. Next, the polymerase (AmpliTaq Gold DNA polymerase) separated the fluorescent dye from the respective probe which was attached to the complementary sequence and from the non-fluorescent quencher via a polymerase chain reaction, wherein the fluorescent dye at the 5′ end of the respective probe was able to release fluorescence. According to the different fluorescence which was detected, different genotypes were able to be identified. In the experiment, the TaqMan(R) Genotyping Assays rs12320939 C_{—}31833529_{—}10 was used to detect rs12320939, the TaqMan(R) Genotyping Assays rs12313273 C_{—}31833531_{—}10 was used to detect rs12313273, the TaqMan(R) Genotyping Assays rs7135617 C_{—}248428_{—}10 was used to detect rs7135617, the TaqMan(R) Genotyping Assays rs6486795 C_{—}1596513_{—}20 was used to detect rs6486795, and the Custom Taqman(R) SNP Genotyping Assay Service rs712853 was used to detect rs712853.

The real-time polymerase chain reaction machine (ABI 7500, Applied Biosystems Inc, Foster City, Calif.) was used to perform the polymerase chain reaction and genotype determination in the invention. 2.5 μl of TaqMan universal Master Mix, 0.0625 μl of 40× TaqMan Genotyping Assay primer-probe Mix, 1 μl of DNA and 1.4375 μl of d. d. d. water constituted each sample with a total volume of 5 μl. Each mixed sample was placed in a 96 well reaction plate. The polymerase chain reaction for each sample was performed in the real-time polymerase chain reaction machine with the reaction conditions as follows: 95° C. for 10 minutes; 95° C. for 15 seconds and 60° C. for 1 minutes for 45 cycles. The software designed by ABI was used to determine the genotype according to the fluorescence intensity detected.

(d) Statistics Analysis

SAS software vision 9.1 was used to perform the statistics analysis in the invention. Chi-square Goodness-of-fit test of the Chi-square test was used to determine whether each tagging single-nucleotide polymorphism met the Hardy-Weinberg equilibrium (HWE) and when the p value was greater than 0.05, it indicated that there was no deviation in the genotype distribution. The chi-square test was used to determine whether there was significant difference between the distribution for each genotype and the allele of patients suffering from kidney stones and those of the healthy control group. Moreover, the chi-square test was used to determine whether the difference between recurrence of kidney stones in patients suffering from and/or once suffering from kidney stones, with different genotypes, was significant.

2. Relationship Between the Single-Nucleotide Polymorphism of the ORAI1 Gene and Development of Kidney Stone

(a) Kidney Stone Patient Group and Healthy Group

A case-control study of 136 kidney stone patients and 500 healthy people were selected for the study of the invention, respectively. The detailed information for the kidney stone patient group and healthy group are shown as Table 1.

TABLE 1
Detailed information for the kidney
stone patient group and healthy group

| | Case (n)(%) | Control (n)(%) |
|---|---|---|
| Numbers | 136 | 500 |
| Sex: male | 83 (61.0%) | 289 (57.8%)^{a} |
| Age: year (mean ± SD)^{b} | 52.0 ± 12.4 | 49.5 ± 15.5^{c} |
| Age range: year | 18-87 | 27-87 |

^{a}P = 0.556;
^{b}mean ± standard deviation (SD);
^{c}P < 0.081

(b) Analysis for Relationship Between the rs12320939 and the Development of Kidney Stone

The rs12320939 genotypes of 136 kidney stone patients and 500 healthy people were studied. The results are shown in Table 2.

TABLE 2
Relationship between the rs12320939
and the development of kidney stone

| | | | | Odds ratio (OR) |
|---|---|---|---|---|
| | | Case (n)(%) | Control (n)(%) | (95% C.I.) |
| Numbers | | 136 | 500 | |
| ORAI1- | TT | 33 (24.3%) | 119 (23.8%) | 1.03 |
| rs12320939 | | | | (0.61-1.74) |
| | GT | 66 (48.5%) | 244 (48.8%) | 1.00 |
| | | | | (0.64-1.58) |
| | GG | 37 (27.2%) | 137 (27.4%) | 1.00 |
| | | | | (reference) |
| | T | 132 (48.5%) | 482 (48.2%) | 1.01 |
| | | | | (0.77-1.32) |
| | G | 140 (51.5%) | 518 (51.8%) | 1.00 |
| | | | | (reference) |

Overall P value=0.9937

Trend P=0.9241

Dominant model P value=0.9641; Odds ratio (OR)=1.01

Recessive model P value=0.9103; Odds ratio (OR)=1.03

(c) Analysis for Relationship Between the rs12313273 and the Development of Kidney Stone

The rs12320939 genotypes of 136 kidney stone patients and 500 healthy people were studied. The results are shown in Table 3.

TABLE 3
Relationship between the rs12313273
and the development of kidney stone

| | | | | Odds ratio (OR) |
|---|---|---|---|---|
| | | Case (n)(%) | Control (n)(%) | (95% C.I.) |
| Numbers | | 136 | 500 | |
| ORAI1- | CC | 12 (8.8%) | 29 (5.8%) | 2.10 |
| rs12313273 | | | | (1.01-4.37) |
| | CT | 69 (50.7%) | 192 (38.4%) | 1.82 |
| | | | | (1.22-2.72) |
| | TT | 55 (40.5%) | 279 (55.8%) | 1.00 |
| | | | | (reference) |
| | C | 93 (34.2%) | 250 (25.0%) | 1.56 |
| | | | | (1.17-2.08) |
| | T | 179 (65.8%) | 750 (75.0%) | 1.00 |
| | | | | (reference) |

Overall P value=0.0058

Trend P=0.0020

Dominant model P value=0.0015; Odds ratio (OR)=1.86

Recessive model P value=0.2030; Odds ratio (OR)=1.57

(d) Analysis for Relationship Between the rs7135617 and the Development of Kidney Stone

The rs7135617 genotypes of 136 kidney stone patients and 500 healthy people were studied. The results are shown in Table 4.

TABLE 4
Relationship between the rs7135617
and the development of kidney stone

| | | | | Odds ratio (OR) |
|---|---|---|---|---|
| | | Case (n)(%) | Control (n)(%) | (95% C.I.) |
| Numbers | | 136 | 500 | |
| ORAI1- | TT | 55 (40.4%) | 175 (35.0%) | 1.27 |
| rs7135617 | | | | (0.75-2.16) |
| | GT | 56 (41.2%) | 224 (44.8%) | 1.01 |
| | | | | (0.60-1.71) |
| | GG | 25 (18.4%) | 101 (20.2%) | 1.00 |
| | | | | (reference) |
| | T | 166 (61.0%) | 574 (57.4%) | 1.16 |
| | | | | (0.88-1.53) |
| | G | 106 (39.0%) | 426 (42.6%) | 1.00 |
| | | | | (reference) |

Overall P value=0.5035

Trend P=0.3039

Dominant model P value=0.6373; Odds ratio (OR)=1.12

Recessive model P value=0.2416; Odds ratio (OR)=1.26

(e) Analysis for Relationship Between the rs6486795 and the Development of Kidney Stone

The rs6486795 genotypes of 136 kidney stone patients and 500 healthy people were studied. The results are shown in Table 5.

TABLE 5
Relationship between the rs6486795
and the development of kidney stone

| | | | | Odds ratio (OR) |
|---|---|---|---|---|
| | | Case (n)(%) | Control (n)(%) | (95% C.I.) |
| Numbers | | 136 | 500 | |
| ORAI1- | CC | 28 (20.6%) | 69 (13.8%) | 2.01 |
| rs6486795 | | | | (1.16-3.48) |
| | CT | 66 (48.5%) | 223 (44.6%) | 1.47 |
| | | | | (0.95-2.25) |
| | TT | 42 (30.9%) | 208 (41.6%) | 1.00 |
| | | | | (reference) |
| | C | 122 (44.9%) | 361 (36.1%) | 1.44 |
| | | | | (1.10-1.89) |
| | T | 150 (55.1%) | 639 (63.9%) | 1.00 |
| | | | | (reference) |

Overall P value=0.0348

Trend P=0.0095

Dominant model P value=0.0233; Odds ratio (OR)=1.59

Recessive model P value=0.0509; Odds ratio (OR)=1.62

(f) Analysis for Relationship Between the rs712853 and the Development of Kidney Stone

The rs712853 genotypes of 136 kidney stone patients and 500 healthy people were studied. The results are shown in Table 6.

TABLE 6
Relationship between the rs712853
and the development of kidney stone

| | | | | Odds ratio (OR) |
|---|---|---|---|---|
| | | Case (n)(%) | Control (n)(%) | (95% C.I.) |
| Numbers | | 136 | 500 | |
| ORAI1- | CC | 13 (9.6%) | 65 (13.0%) | 0.73 |
| rs712853 | | | | (0.38-1.42) |
| | CT | 62 (45.6%) | 211 (42.2%) | 1.08 |
| | | | | (0.72-1.61) |
| | TT | 61 (44.8%) | 224 (44.8%) | 1.00 |
| | | | | (reference) |
| | C | 88 (32.4%) | 341 (34.1%) | 0.92 |
| | | | | (0.69-1.23) |
| | T | 184 (67.6%) | 659 (65.9%) | 1.00 |
| | | | | (reference) |

Overall P value=0.5173

Trend P=0.5962

Dominant model P value=0.9912; Odds ratio (OR)=1.00

Recessive model P value=0.2780; Odds ratio (OR)=0.71

According to the results mentioned above, it was discovered that there are significant differences between distributions for the polymorphism positions, rs12313273 and rs6486795 in the ORAI1, of patients suffering from kidney stones and those of the healthy control group. When the rs12313273 CC genotype carriers were compared to the rs12313273 TT genotype carriers, the odds ratio for kidney stones developing was 2.10 (95% C.I: 1.01-4.37). When the rs6486795 CC genotype carriers were compared to the rs6486795 TT genotype carriers, the odds ratio for kidney stones developing was 2.01 (95% C.I: 1.16-3.48).

3. Relationships Between the Recurrence of Kidney Stones and the Single-Nucleotide Polymorphisms of the ORAI1 Gene

An analysis for the genotype of rs12313273 was performed to 136 kidney stone patients. The number of patients having recurring kidney stones was recorded, the genotypes of all patients and the genotypes of the patients with recurring kidney stones were also recorded, respectively, and the results of the determinations were analyzed, wherein the definition of recurrence is described as follows: a patient having at least two symptom occurrences within a 6 month period, or a patient, after being cured, having an occurrence of new kidney stones. The results are shown in Table 7.

TABLE 7
Genotype frequency of ORAI1 SNP rs12313273 for episodes of stone recurrence

| | ORAI1-rs12313273 | | | | | | |
|---|---|---|---|---|---|---|---|
| Genotype | CC | | CT | | TT | | P value |
| Patient numbers | 12 | | 69 | | 55 | | |
| Numbers of patient having | 7 | (58.3%) | 32 | (46.4%) | 15 | (27.3%) | 0.037 |
| recurring kidney stone | | | | | | | |
| Odds ratio (OR) (95% C.I.) | 3.73 | (1.73-13.59) | 2.31 | (1.08-4.93) | 1.00 | (reference) | |

Table 7 shows that when the rs12313273 CC genotype carriers were compared to the rs12313273 TT genotype carriers, the odds ratio for kidney stones to reoccur was 3.73 (95% C.I: 1.73-13.59).

Therefore, according to all of the analytical results mentioned above, it is clearly shown that the single-nucleotide polymorphism rs12313273 of the ORAI1 gene is able to be used as a biomarker for determining development or recurrence of kidney stones.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for determining a risk for kidney stones to develop in a subject, comprising:
obtaining a biosample of the subject;
detecting the presence of the single-nucleotide polymorphism rs12313273 (C/T) at position 30881 of the ORAI1 gene (SEQ ID No.: 1) in the biosample; and
determining a risk for kidney stones to develop in the subject, wherein if the presence of a C allele of the single-nucleotide polymorphism rs12313273 (C/T) is detected, it indicates that the subject has an increased risk for kidney stones to develop.

2. The method for determining a risk for kidney stones to develop in a subject as claimed in claim 1, wherein the risk for kidney stones to develop in the subject is increased with an odds ratio of at least about 1.4-1.6 when the C allele of the single-nucleotide polymorphism rs12313273 (C/T) is detected in the biosample as opposed to the T allele of the single-nucleotide polymorphism rs12313273 (C/T) being detected in the biosample.

3. The method for determining a risk for kidney stones to develop in a subject as claimed in claim 1, wherein the risk for kidney stones to develop in the subject is increased with an odds ratio of at least about 1.7-1.9 when the genotype CT of the single-nucleotide polymorphism rs12313273 (C/T) is detected in the biosample as opposed to the genotype TT of the single-nucleotide polymorphism rs12313273 (C/T) being detected in the biosample.

4. The method for determining a risk for kidney stones to develop in a subject as claimed in claim 1, wherein the risk for kidney stones to develop in the subject is increased with an odds ratio of at least about 2.0-2.2 when the genotype CC of the single-nucleotide polymorphism rs12313273 (C/T) is detected in the biosample as opposed to the genotype TT of the single-nucleotide polymorphism rs12313273 (C/T) being detected in the biosample.

5. The method for determining a risk for kidney stones to develop in a subject as claimed in claim 1, wherein the subject comprises a mammal.

6. The method for determining a risk for kidney stones to develop in a subject as claimed in claim 1, wherein the subject comprises a human.

7. The method for determining a risk for kidney stones to develop in a subject as claimed in claim 1, wherein the kidney stones comprise calcium kidney stones.

8. The method for determining a risk for kidney stones to develop in a subject as claimed in claim 1, wherein the biosample comprises blood or saliva.

9. The method for determining a risk for kidney stones to develop in a subject as claimed in claim 1, wherein the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

10. A method for determining a risk for kidney stones to reoccur in a subject suffering from or once suffering from kidney stones, comprising:
obtaining a biosample of the subject suffering from or once suffering from kidney stones;
detecting the presence of the single-nucleotide polymorphism rs12313273 (C/T) at position 30881 of the ORAI1 gene (SEQ ID No.: 1) in the biosample; and
determining a risk for kidney stones to reoccur in the subject suffering from or once suffering from kidney stones, wherein if the presence of a C allele of the single-nucleotide polymorphism rs12313273 (C/T) is detected, it indicates that the subject suffering from or once suffering from kidney stones has an increased risk for kidney stones to reoccur.

11. The method for determining a risk for kidney stones to reoccur in a subject suffering from or once suffering from kidney stones as claimed in claim 10, wherein the subject has an increased risk for kidney stones to recur when the genotype CT of the single-nucleotide polymorphism rs12313273 (C/T) is detected in the biosample as opposed to the genotype TT of the single-nucleotide polymorphism rs12313273 (C/T) being detected in the biosample.

12. The method for determining a risk for kidney stones to reoccur in a subject suffering from or once suffering from kidney stones as claimed in claim 10, wherein the subject has an increased risk for kidney stones to recur when the genotype CC of the single-nucleotide polymorphism rs12313273 (C/T) is detected in the biosample as opposed to the genotype CT of the single-nucleotide polymorphism rs12313273 (C/T) being detected in the biosample.

13. The method for determining a risk for kidney stones to reoccur in a subject suffering from or once suffering from kidney stones as claimed in claim 10, wherein the subject has an increased risk for kidney stones to recur when the genotype CC of the single-nucleotide polymorphism rs12313273 (C/T) is detected in the biosample as opposed to the genotype TT of the single-nucleotide polymorphism rs12313273 (C/T) being detected in the biosample.

14. The method for determining a risk for kidney stones to reoccur in a subject suffering from or once suffering from kidney stones as claimed in claim 10, wherein the risk for kidney stones to reoccur in the subject suffering from or once suffering from kidney stones is increased with an odds ratio of at least about 2.2-2.4 when the genotype CT of the single-nucleotide polymorphism rs12313273 (C/T) is detected in the biosample as opposed to the genotype TT of the single-nucleotide polymorphism rs12313273 (C/T) being detected in the biosample.

15. The method for determining a risk for kidney stones to reoccur in a subject suffering from or once suffering from kidney stones as claimed in claim 10, wherein the risk for kidney stones to reoccur in the subject suffering from or once suffering from kidney stones is increased with an odds ratio of at least about 3.6-3.8 when the genotype CC of the single-nucleotide polymorphism rs12313273 (C/T) is detected in the biosample as opposed to the genotype TT of the single-nucleotide polymorphism rs12313273 (C/T) being detected in the biosample.

16. The method for determining a risk for kidney stones to reoccur in a subject suffering from or once suffering from kidney stones as claimed in claim 10, wherein the subject comprises a mammal.

17. The method for determining a risk for kidney stones to reoccur in a subject suffering from or once suffering from kidney stones as claimed in claim 10, wherein the subject comprises a human.

18. The method for determining a risk for kidney stones to reoccur in a subject suffering from or once suffering from kidney stones as claimed in claim 10, wherein the kidney stones comprise calcium kidney stones.

19. The method for determining a risk for kidney stones to reoccur in a subject suffering from or once suffering from kidney stones as claimed in claim 10, wherein the biosample comprises blood or saliva.

20. The method for determining a risk for kidney stones to reoccur in a subject suffering from or once suffering from kidney stones as claimed in claim 10, wherein the single-nucleotide polymorphism rs12313273 (C/T) is detected by using a first oligonucleotide and a second oligonucleotide, and the first oligonucleotide is used for detecting the T allele of the single-nucleotide polymorphism rs12313273 (C/T) and the second oligonucleotide is used for detecting the C allele of the single-nucleotide polymorphism rs12313273 (C/T).

21. A method for using a single-nucleotide polymorphism rs12313273 (SEQ ID No.: 3) as a biomarker for determining the development of kidney stones.

22. A method for using a single-nucleotide polymorphism rs12313273 (SEQ ID No.: 3) as a biomarker for determining the recurrence of kidney stones.


SEQUENCE LISTING (6 sequences)

SEQ ID NO: 1 - DNA, 64695 bp, Homo sapiens
aattcctcaacctccaaaactatgtatattttcagcttgatcaactatccttaaagtgtacctctttgaacaagataccggctgggtgcggtggctcatgcctgtaatcccagcactttgggaggccaaggcaggcagatcacctgaagtcaggagtttgagaccagccttgccaacatggtgaaaccctatctctactaaaaatacaaaaattagccgggcattgtggtgggcacctgtagtcccagctactcaggaggccgaggcaggagaatcgcttgaacccggaaggttaaggttgcagtgagccgagatggcgcagctgcactccagcctgggtgacagagtgaaactgtgtctcaaaaaaaaaaaagtcctgacacactggctcacgcctgtaatcctagcactttgggaggccaagacgagcagattgcctgaactcaggagtttgagaccagcctgggtaacatggtgaaaccccgtgtctactaaaacacaaaaaattagccaggggtcgcagtgtgcgcttgtagttccagctactcggaaggttgaagcagaattgcttgaacccaacaggcggaggtgcagtgagctgagatcatgccactgcactccagcctgggtgacagaggcaagactccgtctccaaaaaaaaaagataccaagtttcttagaggtataaaaaatacgttggtgtgccttggcgaggcgggaacacctttctagctccaaagtccagacccttacccactctattccacactgccacccttattcatgacacctgttgcacacaaactgagtcatggggaccagccatcattgctcgcagctgaggcctaaaaacgcgtaccctgcgtcccctccaccgccaccagccagcagtctgggaggcacccctagctcatgatgccgggtagaggaggggccgctgtttgcatgtgactctagcttcctcactgctaggctgtgcctaattattttatttttatttatttatctatctatttatttatttatttattgagacggagtcttgctctgtcgcccaggctcgagtgcagtagcgccatcttggctcactgcaagctccgcctcccgggttcacgccattctcctgcctcagcctcccaaggagctgggactacaggcgcccgccaccacgcccggctaattttttgtatttttaatagatacggggtttcaccgtgctagccaggatgatctcgatctactgacctcgtgatccgcccgcctcggcctcccaaagtgctgggattataggcgtgagccaccgcgcccggcggctgtggctaattattaggtaactagcgtcgttgggtctccctacctgaaggcctaaaggctggagataagagggctgaaggggagtagagccaggactggtgggcctgggagccaccaatagtcccagaagatcctacggggactttgccttctgctcctgatctctggaatcgattcatcagcctgagcttcgactccagcctctgcatccgcttctcccaaggtgattcctcaatgtctgctctgggcacctcatggatgcatcgcaagggctttagggagtcctctgctcacagggattgacctacccgcttgccaggtctcaagctcttgagagaaagcctgatgctctttttgtcttcacctccatcctaggcactgggtaggaacacagccaagaacgattgcaggatgggtccttccaggacactgacgtctcagcttgcgcactgtgagtccctggacgagttactccacctctctgaacctcctcctcacttgcataatgggaaaaataatggacataggaagatgaaacaagaccttggagaccacatttaccatcttattttccttgttcctttttcttttctttttttgtttttgtttgtttgttttttggagacagggtctcactctgtcacccaggctggcgtgcaatggcgcgatctcagctcactgcagcctctgcctcctgtgttcaagggatcctcccacctcagcctcccgggtagctggcaccacagacgcacaccaccatgcctggctaaatttttttgtatttttagtagaagtggggttttaccatgttggccaggctgttctcgaactcctggcctcaagtgatccgtccacctcggcctcccaaagtgctgggattacaggcgtgaaccactgagcccggccccttgttccctgttctgtttctttttcactgttatcactgatcaccatcgatcatattatctacatatctattagattggcgcaaaagtaattgctgtttttgccattacctccaatggcattactttcagtggcaaaaactgcaattacttttgcgccaacctaatatattgagacagggtctctctctgtcacccaggctgctggagggcagtgacacaatcacagctcactgccatgtccaactcctggattcaagtgatcctcccgcctcagtctcctgattacctgggactacaggcatgtatcaccacacccagccggtttttttgtttgtttgtttttgaaacagttttgcttttgccccccaggctggagtgcaatggcatgatcttggttcactgcaacctctgcctccggggttcaagcgattctcctgtctcagcctcccaagtagctgggattacaggtgcctaccaccacacctggctaatttttggttttgtgggtgtgtgtgtgtgtgtgtgtgtgtgtgtgtgtgtgtgttggagtctggctctgtcacccaggctgctggagtgcaatggcgcaatcttcagctcactgcaacctctgcctcctgggtttaagcaattctcctgcctcagactcctgagtagctgagattacaggtgtccaccaccacgcccggctaatttttgtatttttagtagagacagggtttcaccgtgttggccaggctggtctccatctcctgacctcaggtgatccacctgcctcagcctcccaaagtgctgggactacaggcgtgagccaccacgcccggctcaagtttctctttttctttttttttttttacattttgtagagacagggtctcactatgttgcccaggctggtcttgaactcctgatctcaagcaatcctccctctctggctcccgaagtgcggggataataggcatgaaccactgtgcacagcccatattttatattctacttaactgagtttctttagtttttattcctcccactaggatgtctgttttgtttacctccatatctccagcccctagaacagaacttggttcatggtgggttcagtaactatttgaattaaatgaatagatggacgtaaaatggttagtactgcggtgcccagtaatggtaggtatcaaacttagtctctgtgctccaacagaaagcagtgtaatttaagatgggcacagtgggctgggcgcggtggctcacacctgtaatcccagcactttgggaggccgaggcaggtggatcacctggggtcaggagttcgagactggcctggccaacatggtaaaaccttgtctctactaaaaatacacaaaattagctgggtgtggtggtggtcacctgtaatcccagctacttgggaggctgaggcaggagaattgcttcaacctgggaggtggaggttgcagtgagccaagatcacgccactgcactccagcctgggcgacagagcgagactccgtctcaaaaaaaaaaaaagatgggcacagtggctcccgcctgtaatcccagcactttgggaggccgaggtgggcagatcacccgaggtcaggagttcgagaccagcctggccaacatggtgaaaaaccctgtctctactaaaaatacaaaaattagccgggtatggtggcacacgcctatagtcccagctacttgggaggctgaagcaggagaatcacttgaacctgggaggcagagattgcagtaagccaaaatctggccactgcactccagccagggtgacagagccagactccatctcaaaaaaaaaagaacgaaagaaagagagcagtgtaattggagcagggaaaatgcagcatcgtaataggaggtacaggttaggagctctggctgagaaatcccgtctgtaccatgtaccagctgtgtgacctggaacagaagaatgaattcaccttcctgaggctcagtctcctcatctgtcaaatgcaggtgatgtaggacctcctgggaaggatgtgagcatgcaggagatggtaaatgaaaagcacttagcagagtgtctggtgcataataagtgctcagaaaacactggcctacgctgttcattatagtaatactcagatgagggggacctctgagtcaattaaacagttgctaagcacttaaaatatgtcacgggccagcgagtccagtaaacatttattgacttaaatctgtgaactccttaagggaagggactgtgtctacattgtcatgatcccggccccagaagatgctaattaatgtgaaaacatgctggaaaagttacatactgaactcaaatatgagttagaatacagaatagctattagagtaataacacccattgcctaacatgtgccaggcttgctgttgggtgttggagaaagggcagggagcttaaacaaacatgggaacttacagcttagcaggggagacagactttgttctgagaattgcacagatagagaggaaactctgaagttgataaatatcacaaagaagacatatgtggtgctgtgagttccataatatggagggagggagggttgacctggtctcggactggggagggagtgatgcctagagctgagctctgaagaaaagttctttgctaggctgagtgttggggaagtgctctcagcagatgaaatggcagatagaataggccttctgactgaggcacacatgtgctaaagggcgcaaaagaaggcccatatgacacaagcatagtgagctgagggtgcgattgggggtgaaataaactgcaaaaattagtagacactttgggaactagctgggcaatgtctgataaagctgaaggtgtgtgtaacctatgatgcagccgctccacagctgaggacattgttgacagaaatgcaggagtgcactgggaagcatgaacaagaatgatggccctagcagcattgtatatcatcacaaaaaactggaaataggctgggcgcggtgactcatgcctgtaatcccagcactttgggaggccaaggcaggcagatcacctgaggtcaggagttcgagaccagcctggccaacacggtgagaccccatctctactaaaaatacaaaaattagccgggtgtggtggcacatgcctataatcccagctactcgggaggctgatcaggagaactgcttgaacccgggaggcggaggttgcaatgagccaggatcgtaccactgcactccagcctggggcaacagagcaagactttgtctcaaaaaaaaaaaaaaaaaaaaaaaaaagacaggttcaggcctggtgtggtagctcacatttgtaatgccagcactttaggaggccaagacaggagaatcgctttagtccaggagtttgagaccagcctgggcaacttagtgagactctgtctctgcaaaaaaaaatgtaaaagtatgggcgcggtggctcatgcctgtaatcccagcattttgggaggccgaggcaggtggatcacctgaggtcaggagttcgagaccagcctggccaacatggcaaaaccccatctctactaaaaatacaaaaatttgctgggcatggtggtgtgtgcctgtaatcccagctactcaggaggctaaggcaggagaatcacttgaacctgggaggcggaggttgcagtgagccaagattgcaccattgcactccagcctgggcaacaagagcaaaactccatctcaaaaagaaaaatgaaaaaaattagctgggtgtggtggtgcactcctgtggtaccagtttctctggaagctaaggtgggaggatcacttgagcccacaaaatcaaggctgcagtgagccatgatcgcaacactgcactccagcctgggcaacagagtgagaccctgtctccaaaaaaaaaaaaaaaaaaaaattctgggcacggtggttcacgcctgtattcccagcactttgggaggcccaggcaggtggatcacttgaggtcaggagttcaagaccagcctggacaacaggctgtattttgtattttgtctattaaaaatagaaaaattagctcggcatggtggcgtgtgcctgtaattccagctacttgggaggctaaggcagagttacttaaatctgggaggtggaagttgcagtgagctgagatcgcgccactgcactccagcctgggcgacagggagactccatctcaaaaaaataaaaaaataaaaaatacaggcaggttcttgcaacatgtatttcctgtgtaccagaccttgtgttagtccttccttcatgcattcaacatgtattaaacacctactgtgtgccaggcctgatgcctgtaatttattcatacattatttattcaatccaagcaggatttatggagtgcttaggtaatgctaagctctgagcttgtcattcaatgcaatgtatgtttatcaagcagctactgtatacagctgtcccaatcagtgcctgggtacacagtagttcaataaatgcctgaatgaatgaacgccctcaaggagcccccagtcttcctgctaacatggcctgtaaataaataactctaacacacaaagataaggctagaaggtgggtgtgtttagagtgcagagggccccttatcacctgtactgccaggtacctttctcagggccatatcttctcacctgggaaccagctctttgaaggtgaggccaggctggcccctagctcacctgcctgtccatccccgggcacccatggaggactttgcctgcagctcccactcaatcattgcctgctgtctaattacggccgcttccgtgaccgcagttgttccttccttcatacaccaggcaagagggcagcagctgagttctaaccctggagctctggcaaagggcctgcacttctgacaggtgccctccctcagatttggcctgattttccatctatgaaatgaaagatttgattctcctcctccttgacttcttaacaccttgcctggactcctataatggcaagttaattttgaaaaattatagaggtaatccatgatctaattttgcaaaaacccagacaatgcatagaaatcattgaagatgggccaggcgtggtgtctcacgcctgtaatcccagcactttgggaggccaaggcgggcggatcacctgaggtcaggagttcgagaccagcctggccaacatggcgaaaccctgtctccactaaaaatacaaaaattagccaggcatggtggtgcatgctagtaatcccagctactcaggaggctgaggcaggggagtcgcttgaacccaggaagcggaggttgcagtgagccgagatcatgccgttgcactccagcctgggcaacagagtgagacttcatctctaaaaaaataaataaataaataaataaaagaaatcattgaagactcaaatcttcactagagtcacaaagctttgcacaatccaaaactttccttccttccttcttttttttatttatttatttattttattttatttttttgagatggagtttcgctcttgttgcccaggctggagtacaatggtgtgatctcggctcactgcaacctccacctcccaggttcaagcgattctcctgcctcagcctcccgagtagctgggattacaggcatatgccaccacacccagccaattttttgcatttttagtagagatggggtttctccatattggtcaggctggtcttgaactcccaacctcaggtgatccgcccacctcggcctccgaaagtgctgggattacagacatgagctatcgcgctcagcctcctcctctttaactgtggtaaaatatattctgcaggttttttttgtgatggttgtttgtttgtttgtttgtttgtttgtttgtttgtttttgagatggagtctcgctctgtcgcccaggctggagtgcagtgacaccatctcggttcactgcaacctcaccttcctggttcaagccattctcctggctcagcctcagagtggctgggactataggcatgcgccgccacaccccactaattgttttgtgttttttgtagagacaaggtgtctccatgttgtccaggctggtctctaactcctaggctcaagagatctgcccacctcagcctcccaaggtgctgggattataggcttgagcaaccatgcctggcctcctttttttgtgggggagacaggatcttgctgtgttgcttaagctggagtgcagtagtgcaatcatagctcactgcagcctcaacctcctgggcttaagcaagcctcttgtctcagcctctcgagtagctggggctataggtggacaccaccacacccagctaatttttaatttttttatagagatgggggtctcaccatgttgcccaggctggtctcaaactcctgtctcaagtgctccacctacctccccaaagtgctgggattacagggcgggagccactgcgtctgactgtatattaccttttattgtggtcttatcatcagttagtttagaacatttacaggatgaactttaataatgtattgattattttggcaagaaaatggctaataatatttgccgtagtaaaactgagtcattagattactttctggagttacttactgccaagcaaagtaataaagactgttccactgtgggaaggtgtgaaaataaataacattacgcgattctgagcttaaaaataggaaataaacttatatgtacggttttaaaaataataatataggggccgggagcggtggctcatgcctgtaatcccagcactttgggaggccgaggcaggttgatccaaggtcaggagatcgagaccatcctggccagcatggtgaaaccttgtctctaccaaaaatacaaaacttagccaggcatggtggcacacacctgtagtcccagctacttgggaggctgagacaggagaatcgcttgaacccaggaggcttgcagtgagccaagattgcaccactgcactccagcctgggcgatagagggagactccatcccaaaaaaaaaaaaaataatatatatatatatatatatatatatacacacacacacacacacacacaattattactatcgacttaagagagaaaacattaacagtttcatagcagtgctattgacaacaaccacaaaggtgaaaatgacccaaatgtccatcagtagataaatggaaaaataaaacatggtatatggaatataatataatatggaatatgattcagacttaaaaacaaaggaaattctgacacatgctgcaacaagataaaccttgaagacatgctaagtgaaataagccagacatgaatggacaaatatggtatgattccattaatatgaagtacctagagtatttcaattcatggaaacagaaaacagaatgatgggcccaagggctgggcgtggaggcttatgcccgtaattccagcactttgagaggccaaggcgggtggatcatctgaggtcgggagttcgagaccagtctgaccaacatggagaaaccctatctcatggccgggtgtggtggctcatgcctgtaatcccagcagtttgggaggccgaggcgggctgatcacctgaggtcgggagtttgagaccagcctgacctgcagggagaaaccctgtctctactaaaaatacaaaattagccaggcgtggtggcacatgcctgtaatcctacctactagggaggctgaggcaggagaatcgcttgaacccgggaggcggaggttgcggtgagccgagatcgtgccattgcactccagcctgggcaacaagagcgaaaccccatctcaaaacaaaaaaaagagagaaaaagaaaacccatctctactaaaaatacaaaattagctgggcgtggtggcgcatgcctgtaatcccagttactggggaggctaaggtaggagaattgcttgaacccgggcagcggaggttatgatgagccaagattgtgccattacactccagcctggacaacaagagcgaaactccatctcaaaaaaaaaaaaaaaaaagagtcaggcgtggtggcgcgtagctgtaatcccagatacttgggagggtgagacagacgaatcacttgaacgtgggaggcagaggttgcagtgagccgagactgagcgactgcatgtcagcctgggcgacagagtgaaactctatctcaaaaaaaaaaaaaaaagaaagaaagaaagaaaagagagaaagaaaacagaatagaatatagaatagatataatggtggtggccaggggctggatggagggggatattctggggagttaggtttaatggggacagactttttgtttgagaggatggaaaagttctagacgtggatggtggtgatggttgtacaacagtgtgaatatgcttaatgtcatggagctgtacacttaaaaattgttacaatgataaattttatgttacatatattttgtcacaattaaaaaaagcagcagtactcactcactttggcagcacgtatactgaaattgaaaccgcacatagcactttagcatggcccttgcgcgagaatgacaagcagattcatgaggctgagacattccatgttaaaacagcagcaacagaagcccagcaatcccagtgtctctttgaaattctccatggccgggcgtggtggctcacgcctgtaatcccagcactttgggaggccaaggtgggtggatcacgaggtcaggagatccagatcatcctagctaacatggtgaaaccccatctctactaaaaatacaaaaaaaatttagccgggtgtggtggcgggcgcctgtagtcccagctactcaggaagctgaggcaggggaatggagtgaacccaggaggcggagcttgcagtgagccgagatggcaccactgcactccagcctgggcaacagagtgagaccccatctcaaaaaaaaaaaaaaaaagaaattctccacggacccatcccagtctcttcctcctaaccccctgaaagtactagtatatttttgtatttatcgcttattgtaccattgcagggtttttttgtttgtttatttgtttttgagatggagtcttgctctgtcacccaggctggagtgcagtggcgcgatctcagctcactgcaacctctgcctgccgggttcaagtgattctcctgcctcagcctcccgagtagctgggattacaggtgcccgccaccatgcctggctaatttttgtattttttggtagatatggagtttcaccatcttggccaggctggtcttgaactccaggctggtcttgaactcctgacttcgtgatccacccgcctcggcctcccaaagtgctgggattataggcatgagccagcacgcccggcctttttttaaattttttttcgagacagagtcccactctatcacccaccctggagtgcggtagcgcagtctcgtttactgcaatcccctcctcccaagttcaagggattctcctgcctcagcctcccaagtagctggcattacagctgcgcaccaccacacccacctaatttttgtattttttagtagagacggggttttgccatgttagcccagctggtcccaaactcctgaccgcctgcctcagcctctcaaagtgctgggatcacaggcgtgagccaccgtgcgctggcctagtttttatatatattgttttctttggcatgttctttttgttttgaatcattcacttgtttcctttttatttttagagatgggggtctccctctctcacccaggctggagtgcactgatgcaatcacagctcactgcagcctcaaactcctaggctcaagggatcctccttccttagcctccccagtagctaggaccacaggtgcaagccagcatgcctggccaattttttacttttattttttgtagagacagggtttcgccatgttgcccaggctggtcttgaactcctggcctcaaaatatctccctgcctctgcctcccaaagtgctgggtttacaggtgtgagccaatgcacccagccatttcttcttttttgatttaaaaatatttaattgataatggccaggcgtggtggctcacatctgtaatcccagcactttgggaagccaaggcgggtggatcatgaggtcaggagttcaagaccagcctggccaacatggtgaaaccctgtctctactaaaaatacaaaaattagccgggcgtggtggtgcgcgcctgtaatcccagctactcaggaggctgaggcaggagaatagctcgaacccgggaggcagaggttgccgtgagccgagatcgtgccattgcactccagcctgagacagagcaagactctatctcaaaaaaaaattttaactgataaattgaattgtatatattcaagatgtacagtgtgatgagttgaaatccattgtgtcctgattaccatggtcatattgattaacgcatatccatcactcccatagtttccattttacttgtcctgtctttgaattttatgtaaatagacttgatagtatacatatacaattgtccgaatgttagtgtccccccaaaattcatgtgttggaacctaatacccaatgcaatagtattaagagataggtcctttgggaggtgattaagttataaggttctgcgtgtgtgaagggattagtgccctcataaaagcagctcgagggggctatgtgctgtggctcatgcctgtagtcccagaactttgggaggctgaggtaggcagattgcttgagcccaggagttcgagactggcctgggcaaacatggggagaccccgtctcactacacaatattagccggttgtggtggtgcattcctgtggtcccagctacccaggatgctgaggtggaaggatcacctgagcctgggagaatgaggctgtagtgagctgtgattgtaccactacacttcagcctgggcgacagagtgagatcctgtctcaacaacagcaacaaccaaataaataaataaaagaggtccccagcctggccaacatggcgaaatcctgtgtctactaaaaatacaaaaatgtgctaggtgtggtggtgcatgcctgtgatccccaagggaggagaatcacttaaatccagaagctggagcttgcagtgagctgagatcatgccattgcactccagcttggacgacagaactgaccttgtctggaaaagaaaaaaagagacccgagtggagaagctagcccctttccaccatggaagatacagcaacagggtgccatttttgaagtaaactaagtcctcaccagagaccgagtctgctggtgacttgaccttgaacttcccagcccccagaactatgaccaatacattgctgttgttttatttatttatttattttattttatctgagacagagtttcactctctcacccaggctggagtgcagtggcttgatctcagctcaatgcaacctctgcctcccaggttcaagcaattttcctgtcccagcctcccgagtagctgggattacaggtgcccaccaccatgcccagctaattttttgtatttttagtagagacagggtttcaacatttttgccaggctggtcttgaactcctgacctcaggtgatccacccacctcagcctcccaaagtgctgggattacaagcgtgagccaccatgcccagcctagttttattatttgtagagatggggtctggccatgttacccaggctgatctggaactcctgggctcaagtgatcctcctgacttgacctccaaatgtgctgggattataggcacctggcttgaaattttgtttgtttgttttgttttgttttgagatgggggcttactctgtcacccaggctggagagcagtggtgcgatctccacccgctgcaacctctgcctcccaggtttaagcgattctgatgcctcagcctcctgagtagttgggattacagtcatgcgtcacaacacactgcctggctaatttttgtatttttggtagtgatggggtttcgccatgttggccaggctggtcttgaactcctaacctcaggtgatctacccgccttggcctcccaaagtgctgacattgcaaacgtgagccaccacactggagagataggcaggaactagatcacatcagtctttcccatgctggggaaggcgcttggatttcatcctaactcagattctcagatgcatggtcactggagggctgtacacagtgtggcaggctgagtaaatgtcccccagggatgtccatgtcttactgtctggcacctgtgaatatgtgaccgtatatggatgaagggactttgcggatgtgatggagtgaaggattttaagattgatcctggattacctgggtgggcccagtgtaaccacaggtgtcctcaggagaggtagaggaggtcagagaagcgatgtgacaatggaagcagagtctggagaggcgtgacctgctttagaaacagaaggggctgcacactaaggaatgcaggcagccagcagaagggaaaaagcaagaaatggattttcccattatctcattggagcctccaaaggaaccagccctgctgtcaccttaattttggcccctgatgattcattttgaacttcttacctctagaactaagataatgaatttgtgtgtgtgtgtgtgtgtgtgtgtttgtgtgtgtgttttgagatggagtctcactctgtctcgcccaggctggagtgcagtgatgtgatctcggctcactgcaagctccgcctcccgggttcaagcaattctcctgcctcagcctcccgagtcgctgggattacaggcacccgccaccacgcctggttaatttttgcttttttttttttttttttttttggagacaaagactcggtcttgtcccccaggctggagtgtgatggcgcaatctcggctcactgcaacctctgcctcctgggttcaagagattctcatgcctcagccacctgagtagctgggattacaggagactgccaccacgcccggctaatttttgaaattttagtagagatggggtttcaccatgttggccaggctggtctcgaactcttgacctcaggtgatctgcccacctcggcttcccaaagtgctgggattacaggcatgaaccgccgcgcccggccaatttatggtaatttcttacagcagcaatgggaaactaatacaagcagagaaaggacatgatcagatttatgtttcaaaaactatttcttgttgctattacagaatgggctttagcgggatcagcagaagcagagagcccgggtgggtaactgcagacgtgtgggtgggcaacaatggcagcccggatgtgatggaatggttgctgctggacggaagagctcagatctcaagaaaatgctaggggaggccgggcgcagtggctcacgcctgtaatcccagcaccttgggaggccgaggcaggtggatcacgaggtcaggagatcgagaccatcctggctaacaaggtgaaaccccgtctctactaaaaaatacaaaaaaaaattagctgggcgtggtggcgggcgcctgtagtcccagctactcaggaggctgaggcaggagaatggtgtgaacccgggaggcggagcttgcagtgagccgagatcgcgccactgcactccagcctgggcgacggagcgagactccgtttcaaaaaaaaaaagaaaaagaaaaagaaaatgctaggggctagaactgactgggtttattgatgaattggatattgtggggaagggtgcagtctaggaaagtgactgcagcgtgactcctggggtgatcagaactacaatttctccagcttctctggggcctcacagagccacactgtgtttgacaggctgttcccaggatgcgggtgtttttatgtgattaggctggaactgttctctgggaaataaatgccctcagtctaaagggcttttacgagccagggcaggggattaaaggcggccgggctgtggagaaatcccagggttaggagacagctgagcatggagacttttaatctaaaccttaaacgtttttattttccactccttgaactcagggtgtcactttaaatacatgtttggcaacatcagctgttctatttttctttttaattatacaaagcgagatcctggaaatggctcagccccaggctgggggaggcaagggaagtttggcaatggtaggaggcagagaaaccaactaaaaatctagtggatttgaaatccttttgagggagcacagaggccaaacttcgagccaccctgaacttggtaagagctgccctgactcaggatttctaaaaggagaccgacagactcaccagcttagaatggagagaggacagatggacacgtctccaagacaaaaatatcaccagccctagcactgttcctgtacctcgaatttattcccaggccctgtcctgttagctcagctcttttcatttcttttcttctttctttctctttctttcttttctttctttccctttcttcttgctctttctttcttttcttttctttctttctctttttctcatttcttttcctctttctctctctctctttttttttttttattagaggcagggttttactatgttgcccaggctggtcttgaactcctgggctcaagcgatcctcctgcctcagcctcccaaagtactgggattacaggtgtgagccactgtgcctggccctgttacctcctttcatcctctgagcagaaaacagctaactaggttgttcatgacatctatggggtactccctactgtgtgccagctgctgggctcacctgcttacacgtgttacactgttgtaggcagtcagctattctataagtaggtgctattattatccccgttttctagatggggaatctggaatctccagagagaacggatggaacccccacaccccagtgttgggcctgcacttctttgcccttggacccctcccttgcctttctctagccttgctccagatcacagggtggccaacctttggcagggtggcttcctgcacagatggaccaactggaggcagtgccaggagcctggtgggtgggaggagcctggtggtgggaggagcctggtgggtgggaggagccaggtggtgggaggaggggaatccagggtgttcctacccctctgcctcaggtgggatctccagctccacctcccaccagacaggtccactgtggctctggcttctgcctgaggactccagtccctcttcggtaccagaaacaccaccccctgcctctgtccctccaaccccagggtgatagcagtcctcctgctggggctaacctgggggctgcccttcccctgcctgccttttcctgtaagtcttccattacccctggagcaattctcaggataaatgctcttagtgtggtaactgtttgtcctggtcagattctcacagatatactaccttcactaaagacttacagatcagtcaattcattcaatctggaaggcagcaatcctctttctctgagcacagcacatcccatgagagggaaggttggagccaccatttactgagcgctgtgctaattgccagacaacttttctttcatgaaacactcataacaacctctttgccaagtcccatcaatattcccctttcacagatggggctcagagaggctggggtgcttgcccaaagtggcacagttacaaactgacaaaactgggattcaaatccaagagtgcctagctccaaactctgcataatgatctaatctcatctgaacacacttgcagccccattagaattcaacttccttggctggctgaagaccccaccccaaatcactccagcctccttgtattcttttcttcttctttttttttttttttttttgagatggagtctcactctgttgcccaggctggagtgcagtggcgcgatcttggctcactgcaagctctgcctcccaggttcacgccattctcctgcctcagcctcccgagtagctgggactacaggcatccgccaccacgcccagctaattttttgtatttttagtagagacagggtttcaccatgttagccaggatggtctcgatctcctgacctcgtgatccgcctgccttggcctcccaaagtcctggcgtgagccaccgctcctgacctgttttttgttttttgtttttttttttttgagacagagtctcactctgtcacccaggctggagtacaatggcctgatctcagctcactgcaacccccgcctcctgggttcaaacgattcttccacctcagcctcccgagtagctgggactacaggcgtacaccaccattcccagctaacttttgtattttttagtagagacagggtttcgccatgttgcccaggctggtctcctgaccgcaggtgatccgcccacctcggcctcccaaagtgctgggattacaggcgtgagccatcacgcccggcccccccaaaaaaatttttttaagtcccacaaactgggtggcttaggagagaaacttattgtctcacagttctggaggccagaagtccaaaatcaagatgtcagcaaggccacggtccctcggaaggaaccagggaaggacatgccccaggccctgctgctcgagctacctggcttgtagacggccgtcttcttgtgtccattcatgtggtcttccctctgtacatgtctgtctgtgtctaaatttcctcctttgtttttgttttttgttttttgttttttttttctgacagagtttcgctcttgttgcccaggctggcgtgcagtggtgcgatcttggctcactacaacctcctcctcccgggttcaagccattctcctaccttagcctcccgagtaactgggattacaggcacatgccaccacgcccggctaattttgtatttttagtagagataagttttctccatgttggtcaggctggtctccaactcctgacctcaggtgatccgcccgcctcggcctcccaaaaggctgggattacaggcgtgagccacggtgcctggctgtttttgtcttttttgagacagggtctcacactgttgccaaggctggagtgcagtgtcacgctcacagctcagtgcagctttgacctcatgggatcaagcaatgctcctgcctcagcctcactagtagcgggaaccccagacaagagccactgctccttgcaaatttcacacttctcctttttataaggatattagtaatattggaagagggctgtatgagtctgttctgtcattgctataaagaaataccaagctgggcacggtgactcacacctgtaattccagcactttgggaggcccaggccagcagatcacctgaggtcaggagttcgggacaagtctggccaaaatggggaaaccctgtctctactaaaactacaaaacattagtcggacgttgtggcaggcgcctataatcccagctactcgggaggctgaggcaggagaatcgcttgaacccaggaggtggaggttgcagtgaggcgagattgcaccactgcactccagcctgggcaacaagcacaaaattccatttagaaaaaaaaaaaaaaaaatgaaaagaaatacctgaggctgggcaccatggttcacgcctgtaatcccagcattttgggaggccgaggcggacagatcatttgaggccaggagttcgagaccagcctggccaacatggcgaaaccccctctctactaaaaatacaaaaattagccgggcgtggtgatgcactcctgtaatcccagctactcgggaggctgaggcaggagaattgcttgaaccagggaggtggaggttgcagtgagccaacattgagccactgcactccagcctggaagacagagcgagactccatctcaaaaaaaaaaaaaaaaaaaatacctaggccgggcacagtggctcacgcctgtactcccagcactttgggaggctgaggcaggtggatcacgaggtcagcagttcaacctgccggtccaagatggtgaatcctcatctctactaaaaatacaaaaattagctgggcgtggtggcaggcacctgtaatcccagctactcgggaggcggaggcagagaattgcttgcacccagaggcggaagttaaagtgagcccagatcgcgccactgcactccagcctgggcaacagagtgagactctgtctcgaaaaaaaaaaagaaaaagaaatacctaagactgagtaatttataaagaaaagaggatgaatacaaaaaaatcagctgggcgtggtggcagtcgcctgtagtcccagctactccggaggctgaagcaggagaatggccgtgaacccgggaggtggagcttgcagtgagctgatattgcgccactgcactccagcctgggcgacagagcgagactccgtcaaaaaaaaaaaaagaaagaaagaaagaaagaaaagaggattaattggctcttggttcggaaggttgtgcaggaaacattgcagcatctgcttctggggaggcctcaagaagcttccaatcatggcggaaggcaaaggaggaaccagcccttcacatggcgggagcaggaggaagagaggggcggggaggtgctatacacttttaaacaaccagatctcactcactgtcatagcaccaagaggatggtgctaaaccattcatgagaaactggccccgtgatccaatcacctctcaccaggctccatctccaacactggggattacaattgaacatgagatttgggtggggacacagatccaaacgaaataagtgcccaccctatgacctcattttaatttgattacctctgtaaaggctgtatttccaaatgaggtcacattcttcgtttttttttgtttgtggtttttttttttttttttagagagtttcactctttcgcccaggctggagtgcaatggcacaatctcagctgactgcaacctctgccttccagtgtcaagagattctcctgcctcagcctcccaagtagctaggattacaacagtcgcttgccttcacacccagctaatttttgtatttttagtagagatcgggtttcaccatgttggccaggctggtctccaactcctgacctcctgatccaccctcctcggcctcccaaagtgctgggattacagatgtgagccaccgtgcctggccacagtcttcttcttttgtttttttttgagacggagtctcgctctgtcacccaggctggagtgcagtggcgtgatctcggctcactgcaagttctgccttccaggttcacgccattctcctgcctcagcctcccgagtagctgggactacaagcacctgccaccatgcctggctaattttttgtatttttttcattagagacgggggtttcaccgtgttagccaggatggtctcgatctcctgacctcgtgatccgcctgcctcggcctcccaaagtgctgggattacaggcgtgagccatcgtgcccagccggccacattcttctttttgaggtggagtttcactcttgtctgccgggctgcagtgcaatggcgcaatcttggctcactgcaacctccacctcctggctactttttgtattttcagtagagatggggtttcaccatgtttgccaggctggtcttgaactcctgacctcaggtgatccgcctgcctcagtctcccaaggtgttgaaattacaggcgtgagccactgcgcccagccccaaatgaggtcacattctgaggtactgaggtttaggactgcaatgtcttttttggggggacacagtttaacctttaagagtccaacctgatctcatgtgtctgatctctgcagtgtggcctgctttgctaatagaggatgtcagaccgtccaggaaaatgatcctgctgacacccaccaagttaagggatatagtgattctccaaggcatatgtgtattgacaaaatttaccccaacattctgtacagcaaattcagttttgaaaaaggagaactagactaggcgtggtggctcatgcctgtaatccagcactttgggaggctgaggtgggtggatcacctgaggtcacgagttcaagaccagcctgaccaacatgtagaaacctcgtctctactaaaaatgtaaaaattacccgggcgtggtggtgcatgcctgtaatcccagctactcgggaggctgaggcaggagaatcacttgaatccaggaggtggaggttgcagtgagctgagatcatgccattgcgctccagcctgggcaacaagagcaaaactccgtctcaaaaaaaaaaaaaaaaaaagaaagaaagaaaagaaaagaaaaagagaactaggctgggtaggttagctcacgcctgtaatcccagcattttgggaggccatggcaggaggatcgcttgtgtccaggagttttgagactggcctaggcaacatattagatcccatctttataaataataaaaaattagccaggtgtggtggtgtgtgcatgtagtctcagctacttggcaggctgaggtgggagggtcacttgaacccaggaggtcaaggctgcagtgagccataatcgtgcctctggactccagtctaggcaacagaatgaaacttcatctcaaaacaacaacaacaaaaaagaagctggtcacagtggctctagcctttaatcccagcactttgggaggctgaggcaagcggatcgcttgagcccaggagttcaagaccagcctgggcaacgtggcaagaccccctgtctctacaaaaaaaaaaaaaaaaaaaaaaacacaaaaattagccagatgtggtgatgcaggcctgtagccctagctacttgggaggctgagctgagaggatcacttgcacccgggaggcagaggttatagtgggctgagattgtgccactgcactccagtctgggcaagagaccaagacccctgtgaaaaaaaaagaaggaaggaaggaagggaggaagggagggagggaacaagttgaaggtcttacctctacctgactttaagacttattttgaggccgggcgtggtgactcatgtctgtaatctcagcactttgggaggctgaggtgggtgaataacctgaggtcaggagttccagaccagcctggccaacacggcgaatccctgtttctactaaaaatacaaaaattagctggatgtggcagcaggcacctgtaatcctagctactcgggaggctgaagcaggagaatcgcttgaacccgggagccgaggttgcagtgagcccagaacgtgccattgcactccagcctgggcaatagagagagactctgtcttaaaaaagagacttttttttttgagacagggtcttgctctgtcacccaggctgaagtgtagtggccccgtctcggctcactgcaacctccacctcccaggttcaagcaattctcctgcctcagcctcccgagtagctgggactacaggtgcgtgccaccacatccggctaatttttgtatgtttagtagagacaggatttcactgcgttgaccaggctggtctcaaactcctaacctcgtgatccgcccgcctcggcctcccaaagtgctgggattacaggcgtgaaccaccatgcccggccgagacttttttttttttaattttgaggaatttatggggttttttttccccaagacttattttatttttatttatttatttattaatttatttacttttttttttttttttgagatggagtctcactctgtcacccaggctagagtgcaatggcgcgatctcagctcactacaagctctgcctcccgggttcatgccattctcctgcctcagcctccagagtagctgggactacaggcgcctgccaccacgcctggctaattttttgtatttttagtagagatgaggtttcaccgtgttagccaggatggtctcgatctcctgaccttgtgatcggcccgcctcggcctcccaaagtgctgggattacaggcatgagccactgcgcccggccaatttatttacttttgagacggagtcttactctgtcgcccagtatggagtgcagtggcgtgacctcggctcactgctacctccgtctcccgggttcaagggttctcctgccttagcctcccaaatagctgggactacaggtgcgggccaccatgcccagctaatttttgtatttttagtagagatgaggtttcaccatgttggtcagactggtctcaaactcctgacctcaagtgatccgcccaccttggcccccccaaagtactggggttacaggtgtgagccaccacgccctgcccccaagatttactttaaacctagagtaatcaacacagtatctattggcatgagagtagacaaatagatccacagactagaatagagagttcagaaacagtctgtcatgtatagtagtcacgtgatttttgacaaaaatgtccatgcatttcagtggggaaagcttgcttcactcaaaaatctgtatttgattgatgggctataggctcagctacttgagaagctgaggcaggagtgagaatcacttgagtcatgaatttgaggctgtagtgtgcaatgatcacacctatgaagagccactgcactccagcctgggcaacagagcaagaccttgtctctaagaaaaaaacagaaatagaaaaaaatcagtatgacacggatcacagacctaaacatacaagcaaaaacacttttatttttggcggggtgggtggagggtagagacagggttccactatgttgcccaggctggtctcaaacctcctggtctcaagtgatcctctcgacctggcctccccaggtgctgagattataggcatgagccaccatgcctggcctaaaacttttaaaagaaaacacaggtgaatatcttcgtgaccttgtggttggcaaagatttctttttttctttttttttttttttaaatagagtcttactctatggcccaggctggagtgtaatggtgtgatctcagctcactgcaacctccgcctcttgggttcaagtgattcttgtgcctcagcctcttgaatagctgggattacaggcatgtgccaccatgcccggataatttttgtatttttagtagagatgcagtttcaccatgctggccaggctggtctcgaactcctgacctcaggtgatccgtccacctcagcctcccaaaatgctgggattacaggcatgagtcaccgcgtcgtcagcaaagatttcttatccaggccagaaaaagttctcacttttaaaggcatgggcacaaactcagacccaacaaagacttctgaccagtgagggggtaggagtggagggaggaagaatgagcactataagcccagaaactggagagcaaggagacagagttcacactcccctggcatagtctaagctccagagtcagagaaccattctgagaccaacaccagggctcaccaacccgaccccattctgcattttgtaaccagccccctgacagtgtctcacttggtttccagcctccttcaccccaattctttgaccatgaaaaccttcaagatcactcacaaaagctgcctcccagatgtcttgtggagctggcaccagggctggctggcagagggatttcatgcattctgagtcaggacacaaggaaggactgacagggaagctgcccccagctccatacaaggacaacttttgaagtatcagacaaccaatcagacatgctcagcttctagaaggagtgagcttcccatcgccaggggtatgaaaacattgttctagccgggcttggtggtgcatgcctgtggagcccaggagtccgaatgcagcctgggtaacacaatgagacccctgtctccccaccaccaaaaaaaaaaaaaaaactgtggggaaaagttagtaaccatggacccctctaggatcctggggcaggaggcagggatggtccagacaccccacccaagtttttattacttgtgatctgtgcagatggggaaattcaaaccacacagaatattaggagaggggtgaggagcgtgacatagtagctggggaaaggaaaccctcaccaccccaaagccccctctgagtcaccactcccagatccttttgttcctctaaaatcagcaaatctctcctcccagcttccggcctcttccccctccagacctcaggccctcttagctgctaaatgttgtgtgttcctagtcctgaactgggggtttctgggaaccaaggacattgcattgttttgttcattgaacctgtttccattggcagtagccagtgggaggccctggggtgatagcgacgtgattgattgattgattgattttgagagactgagtctccctctgtcgcccaggctggagttcagtggcaggatctcggctcactgcaactttcgcctcctggattcaagcaattcttctgcctcagcctccggagtagctgggattacaggcctggccaccacacccaactaattttttcttttttgagatggagtctagctctgtcacccaggctggagtgcagtggtgtgatctcggctcattgcaacctctgcctcccgggttcaagcaattttcctgcctcagcctcccaagtagctgggactacaggtgtgtgcccccacgctcgactaatttttgtatttttagtagagacggggtttcaccatgttggccagactggtctcaaactccagacctcgtgatctgcccgccttggactcctaaagtgctgggattacaggtgtgagccaccgcgcccggctaattttttcttattttgacacagggtcttactctgtcgcccaggctacagtgcagtggcgtgatctctgctcactccaacctctgcctgccgggttcaagcacttcttctgcctcagcctcccaagtagctgggattataggcatgcaccaccaagcccggctaatttttgtatttttagaagagatgggcttttaccatgttgaccaggctggtcttgaactcctgacctcaggtgatccacctgcctcagcctccgaaagggctggaattacaggcgtgagtcaccgtgcccggcctaatttttgtatttttaatggagacggagtttcgccatgttggccaggctggtctcaaactcctgaccttaagtgatctaccgcctcggcctcccaaagtgttggaattataagcatgagccaccacacctggcccatgattcatttatttatttagttgttttttctttgagacaggatctcgctttattgcctaggttggaggacagtggtgtgatcaccgcagcttcattccaagaagctgggagtacaggctcacgccaccacatctggtttatttatttatttattgagacagagtattgcttttattgcccaggctggagtgcaatggcataatctcatctcatcgcaaactccacctcccgggttcaggcggttctcctgccttagcctcccaagtagctgggattacaggcatgcgccaccacaccagctaatttttgtatttttagtagagacgaggtttctccatgttggtcaggctggtcttgaactcctgtcctctcaggtgatccgcccacctcagcctcccaaagtgctgggattacaggtgtgagccaccgtgcctagtcttatttatttatttatttggagacagagtctcgctctgtcgcccaggctggagtgcagtggcaccatcttggcttactgcaagctccgcctcccgggttcacgccattctcctgcctcagcctcccaagtagctgggactacaggcgcccgccaccacacccagctaatttttttgtatttttagtagagacggggtttcaccgtgttagccaggatggtctcaatctcctgacctcgcgatccgccggtttcagcctcccaaagtgctgggattataggtgtgagccaccgcgcctggcctttttttttttttttttttttaagtagagatgtgctactgcactccagcctgggtgactattgagaccctgtctcaataaataaataaataaataaataaataaataaataaataaataaggccaggcgcagtggctcacatctgtaatcccagcactctgggaggctgaggcaggcagatcatctgagttcaggagttcgagactagcctggacaacatggtgaaaccctgtctctactaaaaatataaaaaattagctgggtgtgctggtgggcacctgtaatcctagttactcaggaggctgaggcagaagaatcgcttgaacctaggaattggaggttgcagtgagctgagattgagccactacgctctagcctgggggacagagcgagactccatcttaaaaataataaaaataaaaataaaataaaatgtttaaatacgttgggcacaatggtttacacctgtaatcccagcactttgggaggctgagatgggtggatcacttgaggtcaggagttcgagaccagcctggccaacatggtgaaaccctgactctactaaaaatacaaaaaattagctgggtgtggtgatggacacctgtaatcccagttatttaggaggctgagccagaataatcggttgaacccaggaggcggaggttgcagtgagccgagatcgagccactgcactgcagcctggggaacagagagagactctgtcctaaaataataataaaataaaataaaatgtttgaataggctggacacagtggctcatgcttgtaattccagaactttgggaggcccagatgggaggatcccttgagcccaggagtttgagaccagcatgctcaacatggggagacgctgtcactattattttttattggagacggagtctccctctgtcgcccaggcttgagtgcagcagcctgatctcagctcactgcaacctctgcctcctgcgttcaagcaattatcctacctcagcttccccagttgctaggattataggcgtgccccatcatacctggctaattttttttgtatttttagtagagatgggggtttcaccatgttagccaggctggtctcgaactcctgaccttaggtgatccacccgcttgggccttccaaagtgttgggataacaggtgtgagccaccgcgcctggccacacttttttttttttttaatgcttaaacagatgaacaggtcaactaaataagctagatgatttcagatgctgtagtgatcacagctataaagggaagaaatcaggatgaggtgacagattgtgacaccggggctacttcaatagagggaaggtctctcaggaggtgctatttgagctgagacacaaatgttgagtgaaaggggtaagaagtacaaaggcgggccatggcgcggtgcgtcacgcctgtaatcccagcactttgggaggctgaggcgggtggatcaccaggtcaagagatcgagaccatcctggtccatatggtgaaaccccgtctctactaaaaataccaaaaatagctgggcgtggtggcgcgcgcctgtagtcccagctactcggcaggctgaagcaggagaattgcttgaacccgggaggcggaggttgcagtgagccgagatcgctccactgcactccagcctggtgacagagtgagactccgtctcaaaaaaaaaaaaaaaaaagtacaaaggccaccagggaaacaaagccagtagggtgagatttcaaattccaaattccccaatttttttttttttttttgagagggactctcgctctgtcgctcaggctggagtgcagtggctcgatctcggctcactgcaagctccgcctcccaagttcaaacgattctcctgcctcagcctccctggtaactgggactagggagtcacctgactaatttttttagtagagacagggtttcaccatgttggctaggctggtctggaactactgaccttgtgatctgcctgcctcggcctcccaaagtgttgggattataggcgtgagtcaccgcgcccggcccccatctttattttaaaaaagccaagaaaaggacaaaaaaccaaacaccgcatgttctcactcataggtgggaattgaacaatgagaacacatggacacaggaaggggaacatcacactccggggactgttgtggggtggggaaaggggggagggatagcattaggagatatacctaatgctaaatgacgagttaatgggtgcagcacaccaacatggcacatgtatacatatgtaactaacctgcacattgtgcacatgtaccctaaaacttaaagtataataataataaaattaaattttaaaaaaaaagccaagaaagggctctgcgggaaccagagacttcttgggggcagaggttttcatcccacaacggttgggtgggggcgcgccctctagtggtgacaagagttacgtttcaccccgccaaccggcaatgtcacatgctcgtcactcattcctgtctctcccatagttattcattcaacaaaaaattactgaggaccgattgtatgccgaggaatattctagatgctgagggtagagctgtgaagctgatagtaaaggtccctgccccggagtttacctcctaggagacattccctagcacagtcctgcatacacccccacctcacctctgctccccaaaattggccttccaacgctccaacagccaaatatccctgcttaaaggtcttggcatgagcagttccctcgccgtcctggaaagctagatcctgtctgtttgcatggcgggctctggtctcagcttaaatgtgacctaagtcagaaaggtgtgatctcacttatttcctgcccctctccacactccactaaaatataatattcgggccgggcatggaggctcatgcctgtaatcgcagcactttgtgaggccgaggcgggcggatctcctgacgtcaggagttccaggctagcctggccaacatggcaaaaccccgtctctactaaaaatacaaaaattagccaggtgtggtggcgggcgcctgtaatcccagctacgcagcaggctgaggcaggagaatcgcttgaaccgggaggtggaggttgcagtgagccaagatcgcgccattgcactcctccagcctgggcgacaagagcaaaaccctgtctcaaaaaaaaaaaaaaaaaaaaaatatatatatatatatatatatatatatatatacacacacacacacatatatatacatatatatgtgtatatatgtgtgtgtgtatatatatatataaaatattcaatgaaggttatcattttcctggaatccctcagcgtagcacgtaatagttgctcagaagacacttgctgaataggtttgaagattcactggtttaaaaaatatcaggtggccgggcacggtggctcacgcttgtaatcccagcactctgggaggccgaggcgggcggatcacaaggtcaggagtttgagaccagcctggccaatatggtgaaaccccatctctactaaaaatacaaaaattagcagggcatggtggcgggagcctgtaatcctagctactcgggaggctgaggcaggagaattgcttgaacccagtaataaaaaaataataaatcaggaaccggtcacctcaatacgtatcagatgctggtgaacacaggtggacaaagtggttaacagacaggttttgtccatctcgttcaccaccctacccagcctcagcacctagaccagtgttggcacccagtgggcgccaaataaacactgcttgaactccagacgtcagccgctctttttcctacagaccttgagccaccttgttccaaaggggatatgggcctcaggaggcgcccagaggtgacctcaggcggcccgacccaggagtccaagctccaggagcagggccacgggagcagctgcggagaggggcggcgccaggagccggagcgggcagccgggcgcttccaggaaaagtggcgggcggcggcgcgccagggaccgtgggcggtgccgtcggagcgggcgggtcacgtgacgcccacaacaacgcccacttcttggtgggcggggcacaggtgggcggggagcatgcaaaacagcccagggcggcggccaatcgcggcgcgcgccgggggtccaggccccggggatccgaggcgccgcccgcgcgcagtctctggtcactgccgcccgggggcttttgccagcggcgccgcgggcctgcgtgctggggcagcgggcacttcttcgacctcgtcctcctcgtcctgtgcggccggccgggtgaggccgggcccgcgtagggggcagtcggcggctgcctccggcggaggtgcctcgcggcgcccgggccggcccgcgcctcggcggcgtgctccatgcatccggagcccgccccgcccccgagccgcagcagtcccgagcttcccccaagcggcggcagcaccaccagcggcagccgccggagccgccgccgcagcggggacggggagcccccgggggccccgccaccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtgagctccggccgccctgccctgccccctctactgtcccggctgctcccgcccgcagccccgacggggctctctgggcgggccgggcgggccagagtgaggggaagagctgggagaaaccgtggtgagcgctgcgcggggggcagaggtgctggacgtgagcactggtgggcgccgagtactccgatctggggtggtgacaagccatatcagagtccgggctcggggtgcttgggtgcctccacatggccatgcctgccccagcaagagtggtagatgcatcccgaagttagctctgtaagaagcccgccccatgtgcgttcaggagcgaaaggagggactcagaagctctaacccagctgggggcgtcaggaggtggggacaaggagaaagaccttgcgctgtcttgtgccccggcacccgaggccggaagttgttagccgtgttcttttatcagctcctagaggggatttcctgacaccccttttccaccgcattttccgagagcctcagcctgggggtttgacaaccagtccccctcagttttttctccagaaacttgccactgagtcactgcgtgtgagggaggagggagggacatgttcccgttgaatgggctctttattttttatttgttccaagacggagtctcgctctgtcgcccaggctggagtgcagtggtgcaatctcggctcactgaaatttccacctcctgagttcaagcgattctcatgcctcagcctcccgagtagctgggattacaggctcgcgccaccacgcccggctaatttttgtgttcttagtagagacggggtttcaccctgttggccaggctgatctttaactctcgacctcgtgatctgccaaatctcggcctcccaaagtgctgggattacaggcgtgagctaccacacccggcccaatgggctctttcttgatctagggcataggcatgctttcctttccccaccatcccaaagagctggagtgcaaagcctctgaggtgtggggtgggtgaggctgcggatgggcctgagaagtggctctggggataccctcaggcttggcatattgaagtttctccgttgcaggggactacataataactacattattaccactcggtgatatcagcctgccatgaatggactctgctccccgcccctcagttctgtagaccagagcactcctgcccccgccaccagtgagctaagcccttggccttgagggagggagtggctgcacccttcgtgttttcttgggaagggagtgtgtctgaaactctccctgctttgccttgcagcagagcctgaattttttaaggcccagagcacgctgggccacagtggaaaggccgggagatggggagtgggaagcagccttccctgctcagttacttgctgcctgtgtgacctcaagcaactcactctccctgtccgggcctcaattcctgaattgatcaaagaaggctgctactcaaaagtttctctgactttgagaataaaattgagggagcccctgaaaggagatcagggagtgtgtttgcataaacagacggcactctcatttttagtttagacaggacggacttctgctctgttaatttcaaatcctaagagaccaggttctgcctgctgcctgaagaatctctaaaagcagctggcatttactgaacactcactcagtgctaggtgccttaactcctgggcttactcaacaaccctctgtggtgattttgtcaaagggctctcccaagaccgcacagccagtctggtggagcgggatttgaacccagctctgctggtgttcagaacttgctcaggcacttgtacccattgcggtgtataggaactctgattgatgtccagccagggatgtgttaggacactaggaaatgagaaatagggcaaaagagtttctttttttttttttttttttttgagacggagtcttgctctgtcacccaggctggagtgcagtggtgcgatcttggctcactgcaagctctgactcctgggttcacgccattctgctgcctcagcctctggagtagctgggactacaggcgcccacaaccacgcctggctaattttttttgtatttttttagtagagacggggtttcaccgtgttagcgaggatcgtctcgatctcctgacctcatgatccacccgcctcggcctcccaaagtgctgggattagaggcgtgagccaccggcgcccggccgcaaaagagtttcttttaagccaaagaaagttttataattggtggtggggagggagctaccagttgtcagcttttccctgcagcgcttggtatttaagataatggttcccccttttcttgtggtcatgagtttatagctgccccaggctccttctcactcctgaggggtgagatccagatgaatgacactttcctgccttctcaagtcccttcttcctttcgaagacaggcctcatggtcaaatgtcacctccttctcttgctaaaaacagtgtggggaagtcaggagactggggcagccagtggcatggagtgggccttggcccacccagtctgtctcttggggacttttattttcccccacctgggtggagaaggcttcctggcactactgatggaatccccaaagttctgcaggcagggaaggaccgaaccccatgatactctccattctgcagttacaagaaggtaactgcatgtgccatgaagttggaaatctataggagggatgggattgggtgcagggtctgaccatgccccactcccacttagacacccagcctgtaagtgagcttgggagaggagagggtgccagcagccccaacgccatggtaacagagtgacaagtttcctggattactctcccttggcttatgagtcagcacaaccaacttcctgcccccagagggcttccttcccagagacctggaaagtcagaggagctgacttgacctttgattgccaagggatggggtcaaccaggtgagattgagggagggtctcccacaaatcctggaggggattgagaggttccccacttcatgggtagaacagcctggccaggtggccgggaagtgttggggatggctggtgggtgacctcaggcaagaggctttacttctttgaagccagttttcttttcttttcttttctttttttttgagacggagtttcctctttgttgcccaggctggagtgcagtagcacaatctcagctcactgcaacctccacctcccaggttcaagcgattctcctgcctcagcctcccgaatagctgggattacaggcacacaccacctcacctggctaatttttgtatttttagtagagacggggtttcaccatgttggccagggtggtctcaaactcctgatctcaggtgatccacccgcctcggcctcccaaagtgctgggattacaggtgtgagccaccatgcctggctgaagccagttttttaatgtgtatggagatggcaggtatcctgctctcccatggactcatatgaaggttcattatgaggccagactgggcccatgaaacacaggaattttcagggtctgatgatgcacaccttgcccggaccctctctgatctgcagatgagattcttgctccacacggcagttctaggaacagcaggcctcaggcgcaccatcctccaaactctaatgagagatacaaagcaagggggcctggatatagcataggatcagcatcccgtagagctccttaagcccagagtactcttgtgggctttgattcaggtcctctgtgcctcagtttatacttcattgagaggcagccagcctcagcctgaccccgtcccctgcaggactggcgagcatatgtggtgatcttgtctgctggggaaggcaggctgtaacattagcctgagctgaagataattgggaaggtcagtctgctgccgtaaacaaaatgctttccatgacctgggatcaattatttttggattatctgcctcctcagttctctgcccgccagcacccttttgttcccaagttctagagctggttcagaagtttcctgtaatttctatgaaaatgagttggaaggaacgactcaagtcataagacactgagcctctgtgacgtgttcagagttcagggaccatgtaaatgagcaagaaattgctattactattcctagtactgatcactgatagtaattattattgctgttgttgattgggctcttgcaataggctagatggtgccccacgtagcatcttgtgtgatcctcctataaaccaggtgagtataggtgatggtcattgagaggaaagacttgcgcatggtcacctggctgggaagctgcagagccctgtggctgtggtgtagcgccattgctgtgggagagaaagcatcagagagtgtaggggaacagagctgagaagctcaagcagggtaaggtcagaacagacacttgagcaagacaacttggttcaaatcccagctctacctcttataagggcaagaccttaggcaagtcatttaacctttctgggcctcagtttcttcatctttaaaatggagtacggccgggcgcattggctcacgcttgtaatcccagcactttgggaggccgaagtgggcgaatcacctgaggtcaggagttcaagaccagcctggccaacatggtgaaaccccatctctactaaaaatacaaaaattagccgggtgtagttgtgcatacctgtaattccagctactcgggaggctgaggcagaagaattgcttgaacctgggaggcggaggttgcaatgagcagaggttgcaccactgcactccagcctgggcaacagagtgagactccatctcaaaaaaaaaaaaaaaaaaaatttttgcagagcacttacagtatctgacccgagtatcccagaaacttgcttacccttttttccattgtgttagggctgctaagggttaactgagaagaatctggttcttgccgagccttgagtgtggtggagttaggacttgagaaagccttcaagtcaaaggaaatggcaggaacagttaaagaagagaaattacagggcctaggggaaaggtacaggggctgtggttggggttgagggtcttcaggaagtagtagagaacggtgaatttagccaggcaagatgatggagggccttgattgggtgggaaatttgaactcttggataggcaacagggagccattgaaggttttagacaggagttacatgatgaattctatgatgacggtggtggtggttgtgattactgtttactgaaaccctgttgtgccaggcatgagcctgaacgcttttctttcattcttactgaatcctcaccatagctcttaaaagtatgtactgtaaacaggaaaactaaggctcctagaagttaagattctccagctagtagaattccagctctggtctttctggctgggagccagttgaagagaaagtctgggtctgtgtcccgctgaccctgtatgcaaattcagcctattcatggcttgtaacaaatttactaagttgcaaccaacacattttgttgtttctttcttttgataactttggtaccaagctcctggtcctttttagaatgagataagggtgaaatgaaacaaagtcgcagcacgcattgcatcttgcaagaccttactgatgtgctgtaaggctaaatgttgattggttaaacttttgtttctgttataaacgcgtgtgtgtctatttatagaaacatttggagtcctggactgtcatggtcaagactttgtaaaccactgcacactaggcctcaataccctcttaagagatggtttgcatttgaggtggggccggcaggctcagacagctccgggtgaccctgcatggttgcggcctagagggcctcatgttccaggctgcagccacccaccctcctaaaagggtggacaagaaaagaactaggccaggggaaggaaggctgtgggacctgaggtcaggcccagcatgggagggctgcctcagagggcctggacttgcaggcgccaggtgcccagggtctccatggagcccagcaggagccaggagagatagctggcaatcaggcgaggtgggctgacggctggtatggtccgtcagggccagccaggccttgcccttcgcagaattatgtaaccctcttcttggcccttgctaaggtttggcatctctagccaaaaccctattggggggatccatggaccagtgtgaggtggagctggtggtcttttgcctttcctatcccttatagggcttgcctcgccaggttcaggcggccagtggctggcggggggctgtttattttgggctgacgggctatatttaccttgtgaagctgcgtgcgatctgatctgatccaatcttgctggggctcagctggggctctggcagtcagctgccagatctgtagggcccctggccctccccctgccacccaaggggagatttccagagcaggaagtatcctggaaaaagtggtggcatttattcctgtgactttttcatcaagctccatgacacatgccaaccgttcctttcttctcacttttttttttcccagttgcaagattttattaaaattttttttgtttgtttcttagaactttatttatttatttatttatttatttatttatttattgatcattcttgggtgtttctcgcagagggggatttggcagggtcatgggacaatagtggagggaaggtcagcagataaacaagtgaacaaaggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccgcccttaatccgtttaaccctgagtggacacagcacatgtttcagagagcacagggttgggggtaaggtcacagatcaacaggatcccaaggcagaagaatttttcttagtacagaacaaaatgaaaagtctcccacgtctacctctttctacacagacacggcaatcatccgatttctcaatcttttccccacctttcccgcctttctattccacaaaaccgccattgtcatcatggcccgttctcaatgagctgttgggtacacctcccagacggggtggtggccgggcagaggggctcctcacttcccagtaggggcggccaggcagaggcgccccgcacctcccggacggggcggctggccgggcagaggctgcaatctcggcactttgggaggccaaggcaggcggctgggaggtggatgttgtagcgagccgagatcacgccactgcactccagcctgggcaccattgagcactgagtgaaggagactccgtctgcaatcccggcacctcgggaggccgaggctggcggatcactcgcggttaggagctggagaccagcccggccaacacagcgaaaccccgtctccacccaaaagatacgaaaaccagtcaggcgtggcggcgcacgcctgcaatcgcaggcactcggcaggctgaggcaggagaatcaggcagggaggctgcagtgagctgagatggcagcagtacagtccagcttcggctcggcatcagagggagactgtggaaagagagggagagggagactgtggggagagggagggggagggagagggagagggagagggcctttcttctcactttgaatcattcttttacacggccccctgaaccctgtggcattgaacttggttccaagtgtgggcacatacagcctgcctaagtggacggagttccaggaatcgtagggtctggtgagggtaatgggctttcttccggagcagggcagcaggccctttcctcttcctcggagagcccagcctctcccctctccccagcccaaaacctcagggctggcgagtgtgggtgtccccgggccgtgtgcgtcccaacagctgcactgctttctggccctggagcctgtgggtgtgacaaatggtgtggaatgtggtggtctctgtcctgtggaggtggccatcaggccaggtgtggcgtggggtacacatccttgtggccgaggtttggagctggagctgcctctaaatatagcaagcgaggccccgagtcggggttggaaggtgggtatgtggggtccctgtctccagtctgagctgagggtatccaggcactatttctcttccatccacttcaagctctgtttttctccttttttcttttcttttcttttttttagagatagagtcctgctctgttgcccaggttgtagtgcagtgatgggatcatagctcactgcagccttgaactcctgggctcaagggatcctcctgccttagcctctcaagtagctgggactggccaggtgcggtggctcacacctgtaatcccagcactttgtgaggctgaggtggggagatcatctgaggtcgggagttcaagaccagcctgagcaacaaggtgaaaccccgtctctactaaaaatacaaaagttagctgggcatagtggcgggcgcctgtaatcccagctactcaggaggctgaggcaggagaatcgcttgaacccgggagggggaggttgcagtgagccaaattgtgccagcgcactccagcctgggtgacagagtgagactccatctcaaaaaacaaaaagtagctggggctacagtcatgcccactatatccagctaatttttttttctttgtgtgtgtgtgtgtgtctgtgtgtgtgtgtgagatagggtctcactttgttgcccaggctggggtgcagtagcgcaatcacggctcattgtagcctcaacctcctgggctcaagggattctctcacctcagccttctgagtagctaggactacaggtatactcactacacccagctaattttttgattttgtagagatgaggtctcactgtgttgcccagggagatctcgaattcctggactcaagtgatcctcctacatcagctccctaaattgctggattacaggtgtgtgcctgctttatgtgtttttctttctttctttctttctttttttttttttttttttttttagacagagtttcgctcttgttgccctggctggagtgcaatggcgcgatcttggttcactgcaacctccacctcctgggttcaagcgattctcctgcctcagcctcctgagcagctgggactataggcacgcattaccacgcccagctaatttttgtatttttagtagagacggggtttcaccatattggccaggctggtctcgagctcttgacctcgtgatccacccacctcggcctcccaaagtgctgggattgcatacgtgagccaccgcgcccggccctgtgtttttctttttaaccaagatttattgagctgctgtagctggccctatgctaaacatctacatatgaaaactacatttaatctcagccaccatgtgagggaggtgctcctttgcccccttcatacccgggctgtggagccaagtggacttggttttgaatccagcagcagagtaaccctttggacagctgacatcacctttctgagcctcagtttcctcgtttgtaaaagaagaataatggtattagtaactcgtgggattctcttttttttttttttttttgagacagaccatgctctgtcaccaagactggagtgtagtggcactatcttggctcactgcaacctctgcctcccgggttcaagcgattctcctgcctcagcctcccaattagctgggattacaggtgcccaccaccacaccccagctaattttttttttttttttgagatggagtctcactctgttgcccacaccggagtatagtggcatgatttcggttcactgcaacctccatgtccaggttcaagcgattctcatggctcagcctcccaattttctgggattacaggtgtgtgccaccacactcagctaatttttttgtattttaagtagaggcagggtgtcaccatgttggcccggctgatcttgaactcctaacctcaagttatctgcctgcttcggcctcccaaagtgctgggattacaggcatgagccaccacacctggcccacccctgactaatttttgtatttttagtagagacagggtttcaccatgttggccaggctggtcttgaactcctggcctcaagtgatccaccctcctcagcttcccagtgtgctgggattacaggcatgagccactgcccctgtcagcctctattttataggtgtggaagctgtggcacagagaggttgagatccttatccaaggtcaacagctttgtaagtggaggagccagtttgcagccagctgtgtggtttgttctgtggtcctgactgcatagagggggagagacactacctcgggggtggctggggcacaggcgaggcaccaagtagggagcagaatgggactgcagacctgtatacaggccccccatacacaccccagcggccagctcagggcctgagccacctaaaggctccagacgtttccagtgagtggatcagtagcagtggacctggggcagctcagcaacagccgctgctgcttgtcctgtcttgggtttcaggttccggggtggcaggccagagcaggaagaggaagtgctgggggtggggaggtggcaggagacactggctgctggagggggctcccctcttccaggtgtaggcaggcctgggggcagagacaagggagctgcaggatgtggccctccatctgccgtgccctggtgacctaccccacgtcttgtgtccagcacccacatgtgctgagataccctcacccgcatggactcagaattctcagggctggctttcggccaccaaagatcttaaacacggtagaaatcctgatggcattgccccaaatttatatgagcttgaaatgttttgtaaacgcctggactgagaagtacatactgccatccactgtgggcattcttagacaaaagcctgtgaatcctgttgaaattgtcagaaaagtcctgtgaataaatgatacttggctgttttaaggagagtgtccaccatcttcagattctcagagatgtccgaccccaagtcagatccagattgtgaccttcatctcgctgcagcccctttttttatgggtgttgaggagggtcttggccaaggttaccggtaaacaggcagttaaggagctggaacatgggtttgccagtttggcctggcgttctttatgcctgagtgtgcccctctggaggatgttgtaaagagactgtggggtggggatgaggttcctaaactgagtaataatgaagctcacttttattgagggtttgttgtgtgccggacactacctgttttacatggattgactctcttaatcctcagaagaagccttcgaggcttgtcccattattcccattttacagatgtgaaaacagaggcacagaaagttgaaagcttgccagagtcatacagcccaatcaagggcaggggagtcagggttcaagccaggcagactgtaaaaggagagcggtggggaaggaaggagtccacacccctacctagggcaggaggggtgaggacaggaattccctgcctgctgggggccaggctagtgtgcggctaatggagaaacttcaacattaggcttgccgctctgagcctgccacatatatagcaaacaccttataaagagtcactgtccttgatggaatgactgccataggtatccgccatctccctacccctgtccctccagccctcatgggttgaccatggttgtgtgcccactgcccgctgctcctggcccagatctgataagctggctcctttgcatgggggtgacttgcaaggcccaggtgaggagcagcgggtgtctggcccggagcatgcccctggtgacctcgggtccacaggtctccaggaactgggggcagctggtgtgtcagccctggggctggggaggagacagccccagggtgtactggccacccagcctgcaccagggctgggcccgtcgcttctgggaagcctgctctgtatgtcaggtcgtgcgctgtgttctcagaagggcctgagtgtgccctgggagggcagccaccacccctctgcatgtccccagagcagggcatgaaggagaagcctctgcttgggtgtgaggatgtggggccagcaggcatgatggctggcgattgctttttttaatgcttttattttaaccatagtaaaatacgcataacaaaatttaccattttgaccactgatggtggtggttggtactaattttaattttattattattattattattattattattattattattattattattgagttagagtctcactctattgcccaggctggagtgcagtggcacgatctcagctcactgcaacctccgccacccgggttcaagtgattctcctgcctcagcctcccaagtagctgggattacaggcacgtgccaccacgcccagctaatttttgtattttcagtagagatggggtttcaccatgttggccaggctggtctccaactcctgacctcaggtgatccacccgcctcaatttcccaaagtgctgggattacaggcatgagccactgggcccggcctgattttattttttttagagacaaggtcttgctctggtgcccagactggagggccatggggcagtcatagctcaccaccacctcaaactcctgggctccagcgatccttccccaccagactcccaagtagctagaactacaggtgtgcaccaccaccccccagctaattaaacaaattttttttatagaggtggggtttctctgtgttgcctggctgctctcgaactcctggcctcaagcagtcctcccaccgcagcctccacaagtattgagattacaggcgtgagccactgtgcctgtcctgcggctggtttctaatacctggctaggtggtaatagcagtgttttgggaggtggttaccatgtgccagctctcatttaattgttaaaataattctatgaattggctgggtgtggtggctcacacatgtaatcccaacactttgggaggctgaggcaggcaggtgggtcacctgaggttgggagttcgagaccagcctggccgacatggtgaaaccccgtctctaccaaaagtacaaaaaagtagcaaggtgtggtggcgcatgcctgtaatcccaggtactcgggaggctgaggcaggaggatcacttgaacccgggagtggaggttgcagtcagccgagatcgcatcactgggctccagcctgggcaaaagagcacgactctgtttggagaaaaaaaaaaaaaaaatcctatgaactactattatatccctattttacggataagaaagtcgaggctcaggccgggcgcggtggctcacacctgtaatcccagcactttgggaggctgaggcgggcggatcacgaggtcagaagatcgagaccatcctggctaacacggtgaaaccccgtctgctaaaaatacaaaaaattagccgggcgtggtggcaggcgcctgtagtcccggctacttgggaggctgaggcaggagaatggcgtgaacccgggaggtggagcttgcagtgagccgagatcgcgacactgcactccagcctagcttgggcaacagagtgagactccctctcaaaaaaaaagaaaagaaaagaaaatcgaggctcagagaggttaagtgactttcccagggttgcacagttagtgagtgccagggctgggattcagatccaggcagccttggctcttgctttctgtagggctttctgccactctcatccacaagtggataggccttactatccccattgtagacatatgaaaactgaggctcggagaggccaagcgactggccagggtcccagagcctgacaggaggagagctaggactgaagagtagtagtgtgggctgggaccgctggcactcatcctgcctgtccccccgcaggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatgagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcctaggcccatgtggtctgggcccttccagtgctttggccttacgcccttccccttgaccttgtcctgccccagcctcacggacagcctgcgcagggggctgggcttcagcaaggggcagagcatggagggaagaggatttttataagagaaatttctgcactttgaaactgtcctctaagagaataagcatttcctgttcttccagctccaggtccacctcctgttgggaggcggtggggggccaaagtggggccacacactcgctgtgtcccctctcctcccctgtgccagtgccacctgggtgcctcctcctgtcctgtccgtctcaacctccctcccgtccagcattgagtgtgtacatgtgtgtgtgacacataaatatactcataaggacacctccttcccgtgtcttgtatttgttgggcctgggctactgctcaccctggttaggtgagcctctaggaaaacttaaaacaaattttaagccaggtatggtggcacatacctgtggtctcagctattcaggaggccaaggcaggaggatctcttgagcccaggagtttgagaccccatctcaaacaaaaaatacaaaaattagccagccacggcgcctgcacttccagctcctttgagagactgaggcaggaagattgcctaagcccaggaggccaagtctgcagtgagctatggtaacaccactgcactccaacctgggcaacagagggagactctgtctctaaaaaaatagaaaaatttgccctgcatggtggctcacgcctgtaatcctagccctttggaaggccaaggcgggcagatcacttgaggtcgggagttcgagaccagcctgaccaacatggagaaaccccatctgtactaaaaatacaaaattagctgggtttggtggcgcatgcttgtaatcccagctactcgggaggctgaggcaggagaatcgcttgaacccaggaggcggaggttgcagtgagctgagatcgcgccattgcactccagcctgggcaacaacagtgaaactccgtctcaaaaaaaaaagaaaaaaaaattcaacattttattttgagaaatttcaaacctacaaaaagttgcaggaatagtgtctatctgaaatacatattcagtcttttctttagaagtcttttttttttttttttttttaaatagagcctcactctgtcacccaggctggagtgcagtggcgcgatctgtaaaatcactgtgagcactgacttagcacatactggaccgttgctcctaggagaaatacagggttgcgttcctgtgagctttggtcgtgatattttcatcagctgatcaatatgtaatcttgttttatgtgtatttctgtttaaagattcatatatatatgtgtatatatatatatggttgagttgttaccgttgaactcacagcccacaggactagaacacatgcctaaataaagtttatctaacatacttgttttctccatatgtatatccatcataaccttcttgcactgaggaatattagataatatatatatgtatttatgtattatacatatatatatatatttttttttttgagacagagttttgctcttgttgcccaggctggagtgcaagggcacgatctctgctcactgcaacctccaccttctggtttctttcttttgtttgaaacaaagttttaccttgttgcccaggctccagtgcagcggcacaatctcagcttactacagcctttgcctcccaggttcaagtgattctcctgcctcagcctcttgagtagctaggattacaggcatgcaccaccatgcccagctgatttttgtgtttttagtagagacggggttttaccacgttggccaggctggtctcgaactcctgacctcaagctatccacccgcctgggcctcccaaagtgctgggattacaggcatgagccactgcgcccagcccaccttctggtttccagcgattctcctgcctcagcctcctgaatacttgggattacaggcaccggctaccacacccggctaatttttttgtatttttagtagaggcaggggtttcgctgtgtgggccaggctggtctggaactcgaccccgttatccacccaccttggcctcccaaagttctgggattacaggcgtgagccactggacccggcctattattatatatatatattttgaaacagtctcactccatcccccaggctggagtgcagtggcacaatctctgatcacatgcagcttccgcttcctaggttcaagtgattcttctgcctcagcctcccaagtagctagaatcacaggagcgcgccaccacacccagcaaatttttgtatttttagtagacgggatttcaccatgttggccgggctggtctcgaactcctgacctcacgttatctgcccgcctcagcctcccaaagtgctgggattacagacatgagccactgctccctgcctattttattttagagacagggtttccctctgtcacccaggctggagtacagtggcatgaccatagctcactgtaaccaccaactcctgagctgaagtgatcctccctgcagtgagctctggtcactccacctcccaagtagctaggaccacaggcacatatcaccatgcccagctttttttttttttttttttttgagacggagtttcactcttgttgtccaggctggagtgcaatggtgcgatctcggctcacagcaatctccacctcccgggttcaagccattctcctgcctcagcctcctgagtagctgggattacaggcatacgccaccacgcctagctaattttgtatttttagtagagatggggtttctccatgttggtcaggctggtctcgaactccagacctcaggtgatccacccacctcggcctcccaaagtgctgggattataggtgtgagccaccgcgcccagctttctttttttttttttgagatagaggtttactcttgttgcctaggctagagtgcaatggtgcgatctcggctcactgcaaagctggcaaagtcacagacactgactgaagactgaggagggactgtgtctaccttccccctgaggcatgtgctgtagttaccggtatggtccttcatgcctaaatacttcctcctaaaactaaggacatttccctacatgacgacaatccagtgatcaaatccaggcaattatcagatagaacattcctgttcagtttcccaaatgtcccagtaatgtcccttctagtgacgttttttccacttgaggattgtgtgtcacaacgagatatcacttcctagtctcctttattatttttattttattattatttttttgagacggagtctcactctgtcgcccaggctggagtgcagtggcatgatcttggctcactgcaaaccccgcctcctaggttcaagcgattcttctgcatcagctgcccgagtagctgggattacaggtgcccaccaccacgccgggctaatttttgtatttttagtagagaaggtgtttcaccatgttggctaggctggtctcaaactcccagtctcaagtgatcccctcgccttggcctttcaaagtgttaggattacaggcgtgagccaccgtgcccagccttagtctcctttaatttggaacaattcctcagccttttttttgtttgtttgtgtttgagacagggacttcccatgtcacccaggctagagggcagcctcaacctcctaggctcaagcaatccttccacctcagcctcctgagtagctgggaatacaggcacatgccactacgcctggataatgtttgtattttttatagaaacagggcctctcttgcccaggctggtcttgaactcctggcttcaagggatcctcccaccttggcctcccgaactgctgagattgcagctgtgagccacagcacccaggctagcctttgtctttaaaaaactttttggctgggctcagtggctcacgcctgtaatcccagaactttgggaggccgaggcgggcggatcacgaggtcaggagatcaagaccatcctggctaacagggtgaaaccccgtctctactaaaaaatacaaaaagttagctgggcgtggtggtggctgcctgtagtcccagctgctcgggaggctgaggcaggagaatggcgtgaacccgggaggtggagcttgcagtgagccgagattgcaccactgcactccagcctgggcgacagagagagagtccgtctcaaaaaaaaaaaagaatacaaaaattagctggtcgtggtggtgcgtgcgtgtaatcccagctactcgggagactgagacaggagaatctcttgaacctgggagctggaggttgcagtgagctgagagcgtgccactgcactccagcctgctggacagagcaagactccaaaaaagaaaaatcattatgtgcagctgctattctacgcactatgcatcaactcatttaaccctcagcacccttatatggtgggtacctttttttttttttttttttttgagacggagtctctcacttttgtcacccaggctggagtgcaatggcgcgatctcggctcactgcaagctccacctcccaggttcccgccattctcctccctcaacctcccgactagctgagactacaggcgcccgtcaccatgcccggctaattttttgtatttttagtagagacggggtttcaccacgttagccaggatggtctcgatctcctgacctcgtgatccgcccatctcggcctcccaaagtgctgggattacaggcgtgagccaccgcgcccggccagtggtgagtactattattatctccattttgcagaaggtaaaattcatgcccgtggaagttaagtaaattgccaggatttgaacctggatagtctggctcagagcctaggccctcacaaagatcttacctgggctgattctggctttgctttaaatggagggccgggtgtggtggctcatacctgtaatcccagcactatggaaggctgaggcagctgtatcacctgaagtcagcagtgtttgagaccagcttgaccaacagggtgaaaccccatctctactaaaaatacaaaaattagccaggcgtagtggagcacacctgtaatcccagctacttgggaggctgaggcaggagaatcatttgaacctgggaggcagaggttgcaatgagctgagatcacggcattgcactccagcctgggcgacaagagcaaaactcggtcccaaataaatatatacatacatacatacatacatacatacacacacacacacacatacatacattaaagaactgttggaagccctagctgcatggggccaccttcttacagagcaacaatgagcagcagctgcactgcagccacactctccccagggcctattttttccacagtccccagcactccctcttgtctcacaccaggccgccttcactcatgtgactttcttggcctcattaggcctttgagtttgagatttctgtctgtcccaaacatctaatatagaaagtggttatgctcacgcctgtaatcccagcactttaggaggatgaggcaggtagatgacttgaacccaggagtttgagaccagcctgggcaacatggcgaaaccctgtctctacacaaaatacaaaaattagttgggcatggtggcacgcgcctgtaatcccagctacttgggaaactgaggctggaagatcgcttgagtctgggaagtagagggtgcagtgatccttgattgtgctactggactccagcctgggcaacagaccctgtctcaaaaaagaaaaatttaaaccaagtttttaattagttttataaaagaatgtagtctggctgaagtgaaatgtgggtggagcatcagcaggcctgatatttctctttctcctatcattgttgtcccaggtgcttcacagaaccccaagactctggagaaacacaatttggaaagtgtggccttaagttcttgtcctgtgcggactgggacacagtgaacggcagcttcatagtctgggtggggaaggggaccctccttttccctccagcagcatttgcagtcctgtgtcacatgctccaggatccacacttcttttttgtttgttttgttttgttttgagacagagtctcattctgtcgcccagactggagtacagtggtgaaatcttggctcactgcaacctccgcctcccgggttcaaacgatcctcctgcctcagcctcccaagtagctgggattataggtgcacactaccacgcctggctaatttttgtattttttggtacagatggaggtttcaccatgtcggccaggctagtctggaactcctggcctcatgtgatccacccgcttcagcctcccaaagtgttgggactacaggcgtgagccaccacgcctggcctaatccacacttttgaagggtatcaagtagatgagggagggacctctatgaacccccactgctccccagagaagtcctggacttttttttttttttttttttttgagaccaagtctcactctgttgcccaagctagagtgcagtggcgcgatttcagctcactgcaacctctgcctctcgggttcaagggattctcctgcctcggcctcccgagtagctgggactacaggcatgagccaccacgcccggctaatttttgtatttttagcagagatgggctttcaccacactggccaggctggtttccaactcctgacctcaggtgatccacctgcctcagcctcccaaagtgctgggattacaggcgtgagctaccgcacccggccgtcctggacatcttttacgtattcccctaggacagccacctccctacttctgtagggatgcaagatccagctggaattatttctgggctaggatcctgttgtcttgggttcttgttcctaaactggccgggactcgactcatcagccaggcttgtcaaatgcaggtttcgaagcccttggaggattctgatttaagtaggtctgggtgtgtctttggaatctatttctaacaagcaccccaggtgctctcacacccaggcaacttagacaaacactcctagttaggagcacagtctctttaacttcatttttatttacattttgaatacataatacattcacatggttccaaacacaacactgaaaagttgttcttcagctcctgttcccaagtccccaattctccccagagacaaccatctttgccagtttattgtgtttaaatctagcgacattttgtacataaacaagcacaccctcttccaacattttttttttttttgagatggagtctcgctctgttgcccaggctggagagcagcggcacgatctcggctcggcgtgatcttggctcaccactacctccgcctcctgggttcaagtgattctcctgcctcagcctcctgagtagctgggattatagatgcactccaccacacccagctaatttttgtatttttagtagagacagggtttcaccatgttggtcaggctggtctcgaactcctgacctcttgatccgcctgcctcggcctctcaaagtgctaggattacaggcatgagccaccatgccctgcccccactttttctttaaaaatggtaaccctttacactcttctgcacattaagaacagctagacctaagtttaagcacagtactagcttgatgacttgtgatcctccctgaacctgtttcctcctctaaaatggcagtaatagcaagacacctcactgttgtcggggagtgccaggagagaatgcttgctgcgaagctgtcagcacacggcctggaatagagtgagtgctccatatctggaagttatgattaattctgcatcctagggtggccaggcggggcttcccagtgccagaggtacagactgagaaccacacccttggtgaggccaccctacttatgacttagggggcagagagaacctcagtaaggtcagtggtgtttattgaggctttactatgtgctatgtgcctcttgctgaggtcttagctagaatcgtggtatttcagggtttctcaacattttttttttttttttttttggagatggtgtctcgctctgtcgcccaggctggagtgcaatggcgcaatctcggctcactgcaacctctgccacccaggttcaagcaattctcctgcctcagcctcccaagtagttgggattacaggtgctgaattcttttttttttttttttttgtagcatggtgatattgcactatgcttaaccgcatccctggtctctactcactcaatgccagtagccttcttcctgcctgttgtgacatcaaaaatgtcatacattgccaaatgtccccaagaaagagtggggctgagggaggacaaaatcctacccacagtttaaaaccaccgccttatctcatcctcacagtaactaaccacagtaaccccaggtggtaagtactgttaatgccccatattgcaagtgaggagaccaacagaacagagtttgggtgacattcctgagaacacacagctccgagattcacgcccaggcactgtgactccagagctcacctgcccattatgcaagaacatctgatcttggctgggagcagtggctcacacctgtaatcccagcactttgggagtccaaggcgggcagatcacgaggtcaggagatcgagaccgtcctggccaacatggtgaaaccctgtctctactaaaaatacaaaaaattagccttggtggcaggtgcctgtaatcccagctacttgggaggctgaggcaggataatcgcttgaaccagggaggcgaggttgcagtgagccgagattgtgccactgcactccagcctgggtgacagagcaagactctgtctcaaaaaaaaaaaaaaaaaaaaaaaaaaggccaggctcgatggctcacacctgtaatcccagcactttaggagactgagcggggcagatcacgaggtcaggagttcaagaccagcctgaccaacatggtgataccccatctctactaaaaatacaaaaattagctgggctcagtggcatgcgcctgtaatcccagctactcaggaggctgaggtaggagaatcgcttgaacatgggaggcagaggttgcagtgagctgagatcgcgccactgcattccagcctgggcaatagagggagactacgtcttaaaaaaaaaaaatcacctggggaattttctgaaagctcaggcctcaccccagatgaattagatgtgaacctgtgaatctctgggcatgggacctgggcttccacagctttgcaaagtttccatgggatttcaaggttgagagccctgtgttaggctgagaagtggtcaggaaattttatgatctccaatcttggcagccagtgtggcaggatacaggccttctctctgggtcatctgggttatctcacaggggacctgccatttctttctctttttcttttcctttcttttatattttaaaatttttttttgtagagtcagggtcttgctatgttgcctaagctggtctcgaactcctggcctcaggcagtcctcttaccttggcctcccaagttctgggattacaggaatgagccactgcacccggcctaagacctgcccttactcccccaagctaaacccaggcagcccagccaaggcaggtcttccctcttctaaacctgaaagccctgctgagccccctcgagtgccccatccttatcccataggcccaggcttataccacttggttattctttattctcatgcattccaggatgagatcagagaatgaagagggtcccgaggacagcctgggagagaagcaccagccttcctattggcaagaaatgacccagtgctgggggtagtgaggggccctatgagggtgccggggtcatgcctggagctgctgctgactcctccagccagagccgcactggtctgagcgatcgggtgacacaggcttgaatttctcctgaagcgtttgtgttctctgcaggggagagagggatggtggagctggcatttgaaagacgctgttaccaatgtcctctcctcaaaccctcacatcatgcccaggaggacttggactggacaagaccccactggacagatgtgtaaatggaggctgaaggccaactgccttactcaagggtgttcagaagccaggagaccacctcctttcaaccccgtgcccagctcagttgtgtggcccagggtgcaaatagaaggtttcaggcaggactggaatgcactggtcttcagtgaaatggtttgtttgtttgtttatttgtttgtttgtttgttttttgagacacagtctcacccagactggagtgcagtggcacagtctcagctcactgcaacctctgcctcccaggttcaagcaattctctgcctcagcctcctgagtagctggaattacaggtgccctcccccacgcccagctaatttttgtgtttttagtacagatggggtttcaccatcttggccaagctagtcttgaactcctgacctcgtgatccacccacctcggcctcccaaagtgctgggattacaggattataggattactgtgcctggcgagttttgttttttttgagacagagtcccattctgtcgcccaggctggagtgcagtggtgcaatctcagctcactgcaaccttcacctcccgggttcaagtgattcccctgcctcagcctctcgagtagctgggactacaggtgcctgccaccacacctggctaacttttatatttttagtagagatggggtttcaccatgttggccaggctggtctcaaattcctgacctcaacttatccgcctgccttggcctcccaaagtgctgggattaaaggcaagagccaccacagccggtcaaaaggggttttgaagaggaagcgtcaaaaaggacacctgggactgtaggaggcaagaagcctgccctcgatggtcttgctcccccactccccgaccccaagccctttggagaccccaggagatctgtgcattgttctgggccctggagcagcctcagccacatcagaacttggcaggtgtgcccacccctcacctggcatcaatctccttcctctgtcttcctgaacatggccaaggcctccgccagcacaccatcagggtctaggattttgacctggagggaaataccaagaagttggtttggagagcatggaggacccaggctgccagccttgactggcgctgcctgccgtgtggggtgctgccagcctcatcaggagccacctcccttgctagggatgccctgactgaaaacaaaaccttgtctgttccctgtctgggcttggctgggcctggcagatctggcctgggcatggctggggatgctggcttctgtctctgtctgggtgtggccacagggccctggtgagggctgcccacctcaggaatggggaactgagtgggctcaggggcctcgtcacggccaaagtcgatcatcgtcccgcatttggccatattgtccacccagaagccttcaaacagctggccgtggtccagatggaagaaacgccccgccccgttcttcatgcctctctcccagcagccctcgtagcggttcccgttctctgggggaaaggacagggaggtgtggtgcagggtacaccaaactaagctagacccccaggggtcgctcttccagagctcagtgcaaatcctaacacattgggaggccaagatgggaggctcataagcccaggagatcaagaccagcctgagcaacatagtgagaccacatctctgcaaaaaaaaaaatttttttaattagctgggcatagtggtgtgcacctatagtcccagctactctggaggctgaggtgggaggatggcttgagctcaagaagttgaggctgcagtgagctgagattgccccacagcactccagcctgagtgacaacctccgcctcccgagttcaagtgattctcctgcctcagcctcctgagtagctgggattacaggtgtataccaccacatgcagctactttttgtatttttagtagagacagggtttctccacgttggccaggctgatctcaaactcctgacctcatgatccgcccgcctctgcctcccaaagtgctgggattacaggcatgagccactgcgcctggctttttttttaaaaattgagatataagttcatataacataagttggccattttgaagtgtatagttcagtggcatttagtacattcacaatgctgtgcaaccattacctctatctagtttcaaaacgttttaatcacccgaaaaggaaaccctgtgcccattaagcagccattccccacacccactaatgtcaccacccccatagtcctaggcaaccattagtctactttctgtctctgtggaggtgcgtgtcctaaaaatttcatataaatagcatcatatatatgtgaccttttgtgactgaggggagagatagggtgctcttcctctcttgtcccacattgacacaagggtcagtgtgggacaagagtgtggggtgtggcctgtctctgtgggaccaaatgggcgtggccagctcgctgccgggggcgtggtcgggtgggcggggccggcgggggtggggcactcactcaggcgcagcatgccctccccgttgggcttgtcgttctcccactgtccctcgtagatgtcgccgttgctgtaatacatgcggccccacccgctgcgctggctgccacaccagtcaccctcataatactccttgggtccgaaaaactggatcccataaccctgaaagtacgaagatgctactactcagggcgccccccaacaccaccaggaaagcccaccgtcttcccaggcacccctagagccacacaccccagcttcttccttcttcctcagccctacgtccaagcggagggtcgtgtcttagcggagtatcctgtcttttttgtttgtttgtttgtttttagacagagatccactctgtcacccaggctggagtgcagtgatgtgatctcggctcactgtaacctccgtcccccgggttcaagtgattctcctgcctcagcctcccgagaagctgggattacaaacacgcatcaccacgccctgctaatttttgtatttttaggagagacagggtttcaccatgttggccaggctggtcttgaactcctgacttcaagtgatccgcccgcctcagcctcccaaagtgctgggattacaggtgtgagccatagtgcccaccgataatgtcttgaaggatctccccatgctctgtttctctctgtccctctgctggcgcctccatgcagccagtcattaaactccctccttactgacccaccctctcctctctcatccccaccacaccatgcgccatgcagcccctacagcgccggaggactctttaaatcgcccatctaggctgggcgctgtggctcaccggctcactttaggaggctgaggtgggtggatcacttgaggtcaggagtttgagaccagcctggccaacatggtgaaaccccatctctaccaaaaatacaaaaaattagctgggcgtgatggcggcgcctgtgatcccagttactggggaggctgaggcaggagaatcacttgaacctgggagatggaggttgcagtgagctgagaatgcacggtagcctaggtgacagggtgagacgctatctcaaaaaaaaaaaaaatcgcccatctgatcatgctcgcccccctcccactcttattttgacccagaaaaatgaacataaacaccatacttagcctggcgtggtggcttacgcctataatcccagcactttgggaggccgaggcagatggatcacctgaggtcaggagttttagaccagcctgaccaacgtggcaaaaccctgtctctattaaaaatataaaaaattagctgagtgtggtggtgggcacctgtaatactagctacttgggaggctgaggcagaagaattgcttgaacccaggagtcggaggttgcagtgagcccagatcgtgccattgcactcccacctgggcaacagagcaagactgtctcaaaaaacaaacaaacaaacaaacaaaaaacaccacacttaggctgggcgctgtggctcacgcttgtaatcccagcactttgggaggccaaggcaggcggatcacaaggtcaggagatcgagaccatcctggctaacatggtgaaaacccgtctctactaaaaatacaaaaaaattagctgggtgtggtggtgggtgcccgtagtcccagctactcgggaggctgaggcaggagaaggccgtgaaccaggaggcggagcttgcagtgagccaagatcgcgccactgtactccagcctgggcaacagagaaagactccgtctcaaaaaacaaaaaacaccacacttagtgtccaaccacagggaaacaaggtctgtttgcagatcacacccgaggaggttgggttgagaagccaagtctactcctcaaaatcctgatttcccacatatccctgagggcccccgcaactcttctcctcactcctgtcccagggcagacacactctgctgttttacatctcgttccctttgctatttttttgcctcccatctttctcccttttttttcttcctggctaactcctagcatcttttagtgtcacctcctacaggaagcctgccctgaattccctctttttgagacaagtcttgctctgttgtccaggctggagtgtagtggcaccaccatagctcactataacctcagcctcctgagatcaagcaatcctcctgcctcagccttttgaaaagttgggactacagatgtgcaccacaacatctagctaattattattattatatagttttatttatttatttatttattttgagatggagtctcactctgttgcccaggctgaagtgcagtggcgtgatctcggttcactgccacctctgcctcctggcttcaagcgattctaatgtctccgtctccctagctgggactacaggtacacaccactatgcccggctaattttttttgtgttttttatttagtagagacgggggtttcaccatgttggccaggctagtctcgaactcctgacctcaagtgatacacccctgtctgcctcccaaattgctgggattacaggcgtgagccattgtgcttagccttctgtcctgaattctctaggctgggcttgatgaccttcctctgggcaccctgtgaaaacatccctcctaataccatattctacgtaaattggttattcagggctttctcttcctacttgccaataaacaatttgggagcagagaacatgaaaccctgggctctaccacttgctagctgtgtgactccaggcaagactgaaactgcctttgcaaaattatgactgagacagtgaaagagatttaatttaactgactccatcttgcttctaacctctaagctgtccatgtccattcctgggcttaagctgaactaactttgggagaaacttatagtttatagtttaaacaaagacagtaacagccctttcccgaagacctccttcttgcctggggactagattgcctttgtaggattaacattagctacaagattataaattatggtttaggagtcatgcagctggaagctacaagattctgatcctcactaaactgctcctaagatcaatgcttaagatattttgcagaccttgcacttgatggatcagctggcactacccaaatcaataaactgatttatgtgatcttgtggctccccacctaggaactgactcagggcaagaagacagctttgactccctgtgattttatctctgaccaatcagcacgcctggcttactctttcccccacccaccaagttatccttaaaaactctgcagccagaatgttccgggagactgatttgagtaataatacaacccagggccaggcgcggtggctcatgcccgtaatcttagcactttgggaggcccaggctggcggatcacctgaggtcgggagttcgagaccagcctggccaatatggagaaactctgtctctactaaaaatacaaaattagcagtgtgtggtaccacatgcctgtaatcccagctactcgggagactgaggcaggagaatcgcttgaacccaggaggcggaggttgcggtgagccgagatcacaccattgcactccagcgtggcgacaagagcgaaactccatctcaaaaaataaataaattaataataataataataaaactcaggtctcccacacagccagttctgtgttaattactctttctctattgcaattcccgtcttgatgaatcggctctgtctggggagcaagaagacagctttgactccctatgattttatctctgaccaatcagcactcctggttcactgtctttcccccacccaccaagttatccttaaaaactctgctgctggaatgttcggggagactgatttgagtaataatacaactcaaggccaggcaccggcaaggtgaaccccttgggcagttacaagacacctcatctcttcgagcttgtttgcttatcaataaaatgggcatgaagatgatgatgatgatgagctgttgctgtgagagttaactgaatattaaatgtgtttcaggccgggcgcggtggctcacgcctgtaatcccagcactttggaaggccgaggcgggcggatcacgaggtcaggggatggagaccatcctggctaacacggtgaaaccccgtctttactgaaaatacaaaaaattagccgggcgtggtggcgggcgcctgtagtcccagcaacctgggaagctgaggcaggagaatggcgtgaacccgggaggcggagctcgcagtgagccaagatggcgccactgcactccagcctgggcggcaggatgagactccctctcaaaaaaaaaaaaaaaaaaaaaagttttaaatatgtgtttcaggctcacagtagcagttgctcagttgcaatactgtttattattagtgtctctgggactgatggtgaggattcctccacacttgtgggagtagatggagggaacctgtgcccagcttgggtgtttccgggctgagcacgcgcctctggctggcttcggccccccgggaccctcctctcctgtcccctgctctcttgctcccacttttcctacctcccttttattcaggcccagcagaaaggggattctcagaaagtgatacctggggaaaagaaagccaggaaacgagggggaaaagggtggaggcgacttcg

SEQ ID NO: 2 - DNA, 52 bp, Homo sapiens
aagagttacgtttcaccccgccaaccggcaatgtcacatgctcgtcactcat

SEQ ID NO: 3 - DNA, 52 bp, Homo sapiens
tcttggcatgagcagttccctcgccgtcctggaaagctagatcctgtctgtt

SEQ ID NO: 4 - DNA, 52 bp, Homo sapiens
caagttctagagctggttcagaagtttcctgtaatttctatgaaaatgagtt

SEQ ID NO: 5 - DNA, 52 bp, Homo sapiens
aaacgcctggactgagaagtacatactgccatccactgtgggcattcttaga

SEQ ID NO: 6 - DNA, 52 bp, Homo sapiens
gctgggcttcagcaaggggcagagcatggagggaagaggatttttataagag